(19) United States
(12) Patent Application Publication
(10) Pub. No.: US 2026/0036594 A1
(43) Pub. Date: Feb. 5, 2026

(54) FIBROBLAST GROWTH FACTOR BINDING PROTEIN 1 (FGFBP1) AS (BLOOD) BIOMARKER FOR THE DIAGNOSIS OF POLYCYSTIC OVARIAN SYNDROME

(71) Applicant: Roche Diagnostics Operations, Inc., Indianapolis, IN (US)

(72) Inventors: Deirdre Mary Allegranza, Küsnacht (CH); Annunziata Di Domenico, Kilchberg (CH); Martin Hund, Horw (CH); Martin Klammer, Muenchen (DE); Anika Mang, Holzkirchen (DE); Johanna Carolina Sillman, Zug (CH)

(21) Appl. No.: 18/997,742

(22) PCT Filed: Jul. 20, 2023
(86) PCT No.: PCT/EP2023/070094

§ 371 Date: Jan. 22, 2025

(30) Foreign Application Priority Data
Jul. 22, 2022 (EP) 22186486.1

(51) Int. Cl. G01N 33/68

(52) U.S. Cl. CPC G01N 33/689; G01N 2800/361; G01N 2800/50; G01N 2800/52

(57) ABSTRACT

The present invention relates to a method for assessing whether a subject has Polycystic Ovarian Syndrome (PCOS) or is at risk of developing PCOS, to a method of selecting a patient for therapy of PCOS, to a method for monitoring PCOS progression or for monitoring response to treatment and to a computer-implemented method for assessing a subject with suspected PCOS, by determining the amount or concentration of Fibroblast Growth Factor-Binding Protein 1 (FGFBP1) in a sample of the subject.

The present invention relates to methods of assessing whether a subject has Polycystic Ovarian Syndrome (PCOS) or is at risk of developing PCOS, to methods of selecting a subject for therapy, and to methods of monitoring a subject suffering from PCOS or being treated for PCOS, by determining the amount or concentration of Fibroblast Growth Factor-Binding Protein 1 (FGFBP1) in a sample of the subject, and comparing the determined amount or concentration to a reference. Further the present invention relates to a computer-implemented method of assessing a subject with suspected PCOS by determining the amount or concentration of FGFBP1 in a sample of the subject, optionally by determining the amount or concentration of a second biomarker and/or an additional diagnostic criterion and comparing the amounts or concentrations of FGFBP1, optionally the second biomarker and/or optionally the presence of the diagnostic criterion to a reference.

### BACKGROUND

Polycystic ovarian syndrome (PCOS) is a heterogeneous gynecological condition that is defined by a combination of signs of androgen excess and ovarian dysfunction. Patients with PCOS can have a range of clinical presentations, which can be of reproductive and/or of metabolic type. Reproductive presentations include irregular menstrual cycle, infertility, pregnancy complications and hirsutism, whereas metabolic presentations include obesity, insulin resistance, metabolic syndrome, pre-diabetes, type-2 diabetes, nonalcoholic fatty liver disease (NAFLD), and cardiovascular factors. These clinical presentations are also associated with psychological disorders, such as anxiety and depression (Escobar-Morreale, H. F. 2018; International evidence-based guideline for the assessment and management of polycystic ovary syndrome 2018).

The symptoms are not specific for PCOS and often patients are diagnosed only after longer evaluations for infertility. For a final diagnosis of PCOS, other conditions or diseases should be excluded such as pregnancy, non-classical adrenal hyperplasia (NCAH), Congenital Adrenal Hyperplasia, androgen secreting tumors. Cushing syndrome, thyroid disorders, or hyperprolactinemia (Escobar-Morreale H F. Polycystic ovary syndrome: definition, aetiology, diagnosis and treatment. Nat Rev Endocrinol. 2018; 14(5):270-284; Teede H J, Misso M L, Costello M F, et al. International PCOS Network. Recommendations from the international evidence-based guideline for the assessment and management of polycystic ovary syndrome. Hum Reprod. 2018; 33(9):1602-1618). Diagnostic tests which may be used to exclude other diseases are e.g.
17alpha-Hydroxyprogesterone (17-OHP) to exclude NCAH (Nordenstrom and Falhammar 2018).
Prolactin to exclude hyperprolactinemia.
Cortisol to exclude patients suffering from Cushing's syndrome.
Thyroid Stimulating Hormone (TSH) to exclude thyroid disorders

PCOS may be caused by a combination of genetic, epigenetic and environmental factors, such as inheritance.

Despite the fact that PCOS is one of the most common endocrine disorders in women, affecting 10% of the women during their reproductive years, up to 70% of the affected women remain undiagnosed (March W A, Moorc V M, Willson K J, et al. The prevalence of polycystic ovary syndrome in a community sample assessed under contrasting diagnostic criteria. Hum Reprod. 2010; 25(2):544-51).

The mostly widely used criteria for PCOS diagnosis are the so-called Rotterdam Criteria. PCOS is indicated, if at least 2 of the following criteria apply: (i) irregular cycles (oligomenorrhea) and/or ovulatory dysfunction (oligo-anovulation, OA), (ii) clinical and/or biochemical hyperandrogenism (HA) and (iii) polycystic ovarian morphology (PCOM) (PCOS Consensus Workshop Group, Fertil Steril 2004; 81:19-25). The first criterion is defined as menstrual cycles with a cycle length of less than 21 days or more than 35 days or less than 8 cycles per year. Clinical and/or biochemical signs of hyperandrogenism, where clinical hyperandrogenism is defined as hirsutism (excess and male-pattern hair growth) and/or acne, and biochemical hyperandrogenism is defined as higher levels of free androgens compared to healthy controls. Clinical hyperandrogenism is also defined as a modified Ferriman-Gallwey score of >8. Biochemical hyperandrogenism may be assessed using free testosterone or the free androgen index (FAI) which can be calculated measuring total testosterone and sex hormone binding globuline (SHBG). PCOM is usually determined according to the “International Evidence-based Guideline for PCOS 2018” using endovaginal ultrasound transducers with a frequency bandwidth that includes 8 MHz. The threshold for PCOM is considered to be on either ovary: a follicle number per ovary of >20 and/or an ovarian volume ≥10 ml, ensuring no corpora lutea, cysts or dominant follicles are present. If older ultrasound technology is used, the threshold for PCOM could be an ovarian volume ≥10 ml or a follicle count of >12 on either ovary.

Another method for detecting PCOM is measuring the Anti-Müllerian Hormone (AMH) in a subject. AMH is a glycoprotein hormone whose expression is critical to sex differentiation at a specific time during fetal development. Further, AMH produced by granulosa cells of growing follicles usually correlates with the number of antral follicles within the ovary. Therefore, serum levels of AMH may be a surrogate biomarker for the antral follicle count/number (AFC) determined by transvaginal ultrasound. Some studies have suggested serum AMH as a biochemical marker for PCOM. In some studies, AMH threshold values for PCOM in women with PCOS were proposed (Nicholas et al. 2014; Pigny et al. 2016; Dietz de Loos et al., Fertil Steril, 2021). However, according to the “International evidence-based guideline for the assessment and management of polycystic ovary syndrome 2018” serum AMH levels should not be used as an alternative for the detection of PCOM or to diagnose PCOS.

A further method for detecting PCOS is a 3-item PCOS criteria system (Indran et al. 2018). In this system, it was proposed that diagnosis of PCOS is made, if two out of three items are present: (i) oligomenorrhea (defined as mean menstrual cycle length >35 days); (ii) AMH above threshold; and (iii) hyperandrogenism defined as either testosterone above threshold and/or the presence of hirsutism (mFG score ≥5). Alternatively, AMH was suggested in combination with hyperandrogenism and oligomenorrhea (Sahmay et al., 2014) or in combination with SHBG (Calzada et al., 2019).

Another method for detecting PCOS is to measure other hormones, such as e.g. luteinizing hormone (LH) and follicle-stimulating hormone (FSH). However, the diagnostic utility of the LH:FSH ratio for the diagnosis of PCOS seems low as only a small percentage of women with PCOS have significantly elevated LH:FSH ratios (Cho et al. 2005). Actually, there is a wide range of LH:FSH ratios found in women diagnosed with PCOS (Malini and George 2018).

The necessity to consider the results of multiple diagnostic tests and the results of clinical examination needs specific expertise which makes it quite difficult for less specialized physicians (such as general practitioners) to diagnose PCOS in clinical routine. For example, the determination of PCOM by transvaginal ultrasound requires adequate ultrasound equipment and the subjective analysis of ultrasound images by a physician. Furthermore, the result may also depend on the specific ultrasound device used in the assessment of PCOM. Consequently, the diagnosis of PCOS based on the Rotterdam Criteria always includes at least one subjective, device- and operator-dependent and error-prone measurement.

In order to evaluate biochemical hyperandrogenism, there must be a well-established normal range for the androgen that is measured. Testosterone is the most abundant measured androgen, in its total, bound and free form. The practice of measuring free testosterone has its limitations. Direct measurement of free testosterone using radioimmunoassay is highly inaccurate, and does not reflect the true values. The assays have high intra- and interassay variability. Alternatively, a greater degree of accuracy, particularly for clinical research, will be obtained by measuring total testosterone concentration using extraction and chromatography, or gas (GC-MS) or liquid (LC-MS) chromatography-mass spectrometry. The diagnostic performance of measuring serum testosterone may be enhanced by the concomitant measurement of SHBG, such that the calculation of free T concentration from the total testosterone and SHBG levels only requires solving a second-degree equation (Azziz R, Carmina E, Dewailly D, et al. Task Force on the Phenotype of the Polycystic Ovary Syndrome of The Androgen Excess and PCOS Society. The Androgen Excess and PCOS Society criteria for the polycystic ovary syndrome: the complete task force report. Fertil Steril. 2009; 91(2):456-88). The definition of HA may differ depending on ethnicity. The mFG score of >8 to diagnose hirsutism in women with PCOS may not be appropriate for the diagnosis in all ethnicities. East-Asian women have a lower prevalence of hirsutism compared to Caucasians, and a score of >5 has been proposed for defining hirsutism in Chinese women. There are also indications that the level of androgens in blood differs between ethnicities, where the Japanese population has a lower prevalence of raised androgens and testosterone is only recommended as a complementary factor in the diagnosis of PCOS in this population (Huang Z, Yong E L. Ethnic differences: Is there an Asian phenotype for polycystic ovarian syndrome? Best Pract Res Clin Obstet Gynaecol. 2016; 37:46-55; Kubota T. Update in polycystic ovary syndrome: new criteria of diagnosis and treatment in Japan. Reprod Med Biol. 2013; 12(3):71-77).

Patients suffering from PCOS can be classified in four different phenotypes, named A, B, C or D (Neven A C H, Laven J, Teede H J, Boyle J A. A Summary on Polycystic Ovary Syndrome: Diagnostic Criteria, Prevalence, Clinical Manifestations, and Management According to the Latest International Guidelines. Semin Reprod Med. 2018 January; 36(1):5-12). Phenotype A is characteristic for patients showing hyperandrogenism, ovulatory dysfunction and/or irregular cycles and polycystic ovarian morphology. Phenotype B is characterized by hyperandrogenism, ovulatory dysfunction and/or irregular cycles. Phenotype C is characterized by hyperandrogenism and polycystic ovarian morphology. Phenotype D is characterized by ovulatory dysfunction and/or irregular cycles and polycystic ovarian morphology.

Currently, there is no specific PCOS medication available. Treatment is symptom-oriented and adapted to personal needs. Therapeutic approaches target hyperandrogenism, irregular cycles and/or ovulatory dysfunction and associated metabolic disorders, such as diabetes. The International evidence-based guideline for the assessment and management of polycystic ovary syndrome of 2018 provides information to support clinical decision making and patient management.

Inconsistent diagnostic criteria, variable provider knowledge, and lack of consensus pose specific challenges for the diagnosis and care of women with PCOS. These factors encourage inaccurate diagnosis with both under and over-diagnosis. This unfavorable diagnostic experience exacerbates affected women and limits timely opportunities for intervention to minimize associated comorbidities, especially during the transition from pediatric to adult care (Witchel S F, Teede H J, Peña A S. Curtailing PCOS. Pediatr Res. 2020; 87 (2): 353-361). Further, timely diagnosis is pivotal to prevent further metabolic complications in affected women, such as e.g. type 2 diabetes mellitus.

In the largest study of PCOS diagnosis experiences, many women reported delayed diagnosis and inadequate information. One-third or more of women reported >2 years (33.6%) and >3 health care professionals (47.1%) before a diagnosis was established. Few were satisfied with their diagnosis experience (35.2%) or with the information they received (15.6%). These gaps in early diagnosis, education, and support are clear opportunities for improving patient experience (Gibson-Helm M, Teede H, Dunaif A, Dokras A. Delayed Diagnosis and a Lack of Information Associated With Dissatisfaction in Women With Polycystic Ovary Syndromc. J Clin Endocrinol Metab. 2017; 102(2):604-612).

An area of particular interest in the diagnosis of PCOS is in women of young age, namely adolescents and young women under the age of 25, when the features of normal pubertal development overlap with adult diagnostic criteria. This makes diagnosis controversial and challenging. Many of the manifestations that are used for diagnosing PCOS may evolve over time and change during the first years after monarche. Normal pubertal physiological changes such as irregular menstrual cycles, acne and PCOM, overlap with adult PCOS diagnostic criteria. In adolescent and young adult women, PCOS is diagnosed when both the OA and HA criteria are fulfilled. The pelvic ultrasound is not recommended to be done in adolescents <8 years from menarche, due to the high incidence of multifollicular ovaries in this life stage (Peña A S, Witchel S F, Hoeger K M, Oberfield S E, Vogiatzi M G, Misso M, Garad R, Dabadghao P, Teede H. Adolescent polycystic ovary syndrome according to the international evidence-based guideline. BMC Med. 2020; 18(1):72). The assessment of an irregular menstrual cycle in adolescents can be difficult. Menstrual cycles are often irregular during adolescence. Immaturity of the hypothalamic-pituitary-ovarian axis during the early years after menarche often results in anovulation and cycles may be somewhat long. However, 90% of cycles will be within the range of 21-45 days, although short cycles of less than 20 days and long cycles of more than 45 days may occur. By the third year after menarche, 60-80% of menstrual cycles are 21-34 days long, as is typical of adults. Young girls and their caretakers (eg, parents or guardians) frequently have difficulty assessing what constitutes normal menstrual cycles or patterns of bleeding. Patients and their caretakers may be unfamiliar with what is normal and patients may not inform their caretakers about menstrual irregularities or missed menses. In addition, a patient is often reluctant to discuss this topic with a caretaker (ACOG Committee Opinion No. 651: Menstruation in Girls and Adolescents: Using the Menstrual Cycle as a Vital Sign. Obstet Gynecol. 2015; 126(6):e143-e146). Hence, in particular for this group of patients, the establishment of a reliable biomarker as an aid for the diagnosis of PCOS or for the identification of patients which are at risk of developing PCOS is of utmost importance. Delay in diagnosing adolescents and young women is often due to unwillingness to diagnose adolescents at risk because of puberty and fear of over- or under-diagnosis. This may lead to long-term complications, such as obesity and insulin resistance, and anxiety or depression. The most recent Guidelines for the diagnosis of PCOS in adolescents and young women have defined oligo-anovulation, irregular menstrual cycle and hyperandrogenism as the criteria to improve diagnostic accuracy in this patient group (Peña A S, Witchel S F, Hoeger K M, et al. Adolescent polycystic ovary syndrome according to the international evidence-based guideline. BMC Med. 2020; 18 (1): 72). The Guidelines also recommend a re-evaluation of the diagnosis in 3 years intervals and a lifestyle change to minimize symptoms and comorbidities associated with PCOS, such as anxiety and depression.

So far, there is no universal biomarker available, either used alone or in combination with the symptoms described above or the hormone levels as described previously, to assess whether a subject has PCOS or is at risk of developing PCOS, and/or to determine response to therapy in a subject with PCOS, and/or to monitor PCOS progression in a subject, and/or to monitor response to treatment in a subject having PCOS.

Therefore, there is an unmet need to establish better diagnostic assays for diagnosing PCOS in young women and adolescents.

### SUMMARY OF THE INVENTION

In a first aspect, the present invention relates to a method of assessing whether a subject has PCOS or is at risk of developing PCOS comprising;
(a) Determining the amount or concentration of FGFBP1 in a sample of the subject, and
(b) Comparing the determined amount or concentration to a reference.

In a second aspect, the present invention relates to a method of selecting a patient for therapy of PCOS, comprising:
(c) Determining the amount or concentration of FGFBP1 in a sample of the subject, and
(d) Comparing the determined amount or concentration to a reference.

In a third aspect, the present invention relates to a method for monitoring PCOS progression in a subject or for monitoring response to treatment in a subject having PCOS, said method comprising:
(e) determining the level of FGFBP1 in a first sample of the subject,
(f) determining the level of FGFBP1 in a second sample of the subject which has been obtained after the first sample, and
(g) comparing the level of FGFBP1 in the first sample to the level of FGFBP1 in the second sample, and
(h) monitoring progression in the subject suffering from PCOS or being treated for PCOS, based on the results of step c).

In a fourth aspect, the present invention relates to a computer-implemented method of assessing a subject with suspected PCOS comprising the steps of:
(a) receiving a value for the amount or concentration of a first biomarker in a sample of the subject, said first biomarker being FGFBP1,
(b) optionally, receiving a value for the amount or concentration of a second biomarker in a sample of the subject,
(c) optionally, receiving a value for the presence or absence of at least an additional diagnostic criterion selected from the group consisting of oligo-anovulation, hyperandrogenism and polycystic ovarian morphology;
(d) comparing the values for the amounts or concentrations of steps (a)-(b) to references for said biomarkers and the value for the presence or absence of the at least one additional diagnostic criterion, and/or calculating a score for assessing the subject with suspected PCOS based on the amounts or concentrations of the biomarkers and the value; and
(e) assessing said subject based on the comparison and/or the calculation made in step (d).

### LIST OF FIGURES

FIG. 1. ROC curve analysis for Fibroblast Growth Factor-Binding Protein 1. Results obtained using Olink proximity extension technology

FIG. 2. Scrum Fibroblast Growth Factor-Binding Protein 1 in PCOS cases (only phenotype A) versus controls. Results obtained using Olink proximity extension technology.

FIG. 3. ROC curve analysis for Fibroblast Growth Factor-Binding Protein 1 for PCOS cases when all phenotypes were combined (phenotypes A-D). Results obtained by ELISA immunoassay.

FIG. 4. Serum Fibroblast Growth Factor-Binding Protein 1 concentrations (pg/mL) of controls versus PCOS cases when all phenotypes were combined (phenotypes A-D). Results obtained using ELISA immunoassays.

FIG. 5. ROC curves for Fibroblast Growth Factor-Binding Protein 1 for PCOS phenotypes A-D when compared to controls. Results obtained by ELISA immunoassay.

FIG. 6. Serum Fibroblast Growth Factor-Binding Protein 1 (pg/mL) in healthy controls and in PCOS phenotypes A-D. Results obtained using ELISA immunoassay.

FIG. 7. ROC curve analysis for Fibroblast Growth Factor-Binding Protein 1 for young PCOS cases (age≤25) when all phenotypes A-D were combined. Results obtained using ELISA immunoassay.

FIG. 8. Serum Fibroblast Growth Factor-Binding Protein 1 (pg/mL) of young controls versus young PCOS cases (age≤25) combined phenotypes A-D. The results were obtained using ELISA immunoassay.

FIG. 9. ROC curves for Fibroblast Growth Factor-Binding Protein 1 for young PCOS cases (age≤25) separated by phenotypes A-D versus young healthy controls (age≤25). The results were obtained using ELISA immunoassay.

FIG. 10. Serum Fibroblast Growth Factor-Binding Protein 1 concentrations (pg/mL) in young healthy controls (age≤25) and in PCOS phenotypes A-D (age≤25). Results obtained by ELISA immunoassay.

FIG. 11. ROC curve analysis for Fibroblast Growth Factor-Binding Protein 1 for PCOS cases when all phenotypes were combined (phenotypes A-D). Results obtained by ELISA immunoassay.

FIG. 12. Scrum Fibroblast Growth Factor-Binding Protein 1 concentrations (pg/mL) of controls versus PCOS cases when all phenotypes were combined (phenotypes A-D). Results obtained using ELISA immunoassays.

FIG. 13. ROC curves for Fibroblast Growth Factor-Binding Protein 1 for PCOS phenotypes A-D when compared to controls. Results obtained by ELISA immunoassay.

FIG. 14. Serum Fibroblast Growth Factor-Binding Protein 1 (pg/mL) in healthy controls and in PCOS phenotypes A-D. Results obtained using ELISA immunoassay.

FIG. 15. ROC curves for Fibroblast Growth Factor-Binding Protein 1 for different PCOS age groups (15-<20, 20-<25, 25-<40) when compared to controls. Results obtained by ELISA immunoassay.

FIG. 16. Scrum Fibroblast Growth Factor-Binding Protein 1 (pg/mL) in healthy controls and in PCOS age groups (15-<20, 20-<25, 25-<40). Results obtained using ELISA immunoassay.

FIG. 17. ROC curve analysis for Fibroblast Growth Factor-Binding Protein 1 for young PCOS cases (age 15-<25) when all phenotypes A-D were combined. Results obtained using ELISA immunoassay.

FIG. 18. Serum Fibroblast Growth Factor-Binding Protein 1 (pg/mL) of young controls versus young PCOS cases (age 15-<25) combined phenotypes A-D. The results were obtained using ELISA immunoassay.

FIG. 19. ROC curves for Fibroblast Growth Factor-Binding Protein 1 for young PCOS cases (age 15-<25) separated by phenotypes A-D versus young healthy controls (age 15-<25). The results were obtained using ELISA immunoassay.

FIG. 20. Serum Fibroblast Growth Factor-Binding Protein 1 concentrations (pg/mL) in young healthy controls (age 15-<25) and in PCOS phenotypes A-D (age 15-<25). Results obtained by ELISA immunoassay.

### DETAILED DESCRIPTION OF THE INVENTION

The inventors of the present invention have identified Fibroblast Growth Factor-Binding Protein 1 (FGFBP1) as a reliable biomarker for diagnosing PCOS in a subject, for determining if the subject is at risk of developing PCOS, to determine response to therapy in a subject with PCOS, or to monitor PCOS progression in a subject having PCOS, or to monitor response to therapy in a subject having PCOS. FGFBP1 can be used either alone or in combination with at least an additional criterion such as hyperandrogenism, oligo-anovulation, PCOM or irregular cycles for the diagnosis, risk assessment and/or monitoring response to therapy in a patient. Further, determination of the level of FGFBP1 compared to a control level can be used to monitor response to a treatment and/or to monitor PCOS progression in said subject.

We show for the first time that FGFBP1 value measured in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, is increased in women suffering from PCOS compared to controls. Further, we show for the first time that FGFBP1 value measured in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, is increased in women suffering from any phenotypes A to D of PCOS. The solution provided by this invention is an immunoassay that detects Fibroblast Growth Factor-Binding Protein 1 in a sample, preferably a biological fluid sample, which is preferably blood, plasma, serum, capillary blood, interstitial fluid, peritoneal fluid, or menstrual fluid and wherein the biological fluid sample is more preferably blood, plasma or scrum. This immunoassay can be used to diagnose women with PCOS also in combination with other clinical and/or biochemical features such as oligo-anovulation and/or irregular cycles, hyperandrogenism or PCOM. Further, measuring the FGFBP1 value can be used to monitor progression of PCOS in said patients and to monitor response to therapy. We show also that the measurement of FGFBP1 values in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, either alone or in combination with an additional diagnostic criterion as described above, is particularly suitable for the diagnosis of PCOS in adolescents or young women under the age of 25 years, in particular under the age of 20 years, in particular 15 to under the age of 25 years, in particular 15 to under the age of 20 years.

There is an unmet medical need for an accurate test for the reliable diagnosis of PCOS. Measurements of FGFBP1 in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, have the advantage of a reliable biological fluid-based test that identifies women suffering from PCOS that is currently not possible. Measurements of FGFBP1 in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, can also be reliably used in adolescent subjects and young women under the age of 25 years, in particular under the age of 20 years, in particular 15 to under the age of 25 years, in particular 15 to under the age of 20 years, for the diagnosis of PCOS. Diagnosis of PCOS in adolescent patients is difficult for the reasons described above and hence, for the first time, we provide an accurate test for the diagnosis of PCOS in the adolescent and young women population. In addition, measurements of FGFBP1 in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, have the advantage to identify if the patient responds to therapy. An additional advantage of measurements of FGFBP1 in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, of a patient is to monitor the progression of PCOS. Furthermore, we enclose a computer-implemented method for assessing a subject suffering from PCOS by measuring FGFBP1 levels in a sample, preferably a biological fluid sample, which is preferably blood, plasma, serum, capillary blood, interstitial fluid, peritoneal fluid, or menstrual fluid and wherein the biological fluid sample is more preferably blood, plasma or serum, and, optionally, with further criteria such as a value for oligo-anovulation and/or irregular cycles, hyperandrogenism and/or polycystic ovarian morphology or with further biomarkers or hormones, for assessing said subject based on the comparison and/or the calculation of the data described above.

As described above, patients suffering from PCOS may show two types of characteristics-reproductive or metabolic type. The metabolic type of PCOS include obesity, insulin resistance, metabolic syndrome, pre-diabetes, type-2 diabetes, nonalcoholic fatty liver disease (NAFLD), and cardiovascular factors. The term “phenotypical” can be used instead of “reproductive”. The term “reproductive” (or “phenotypical”) describes any feature of the phenotype of a female known to be indicative of PCOS. For example, these reproductive characteristics comprise polycystic ovarian morphology (PCOM) and/or clinical hyperandrogenism, such as acne, seborrhea, alopecia, and/or hirsutism. Preferably, these reproductive characteristics comprise polycystic ovarian morphology (PCOM) and/or clinical hyperandrogenism, more preferably acne, seborrhea, alopecia, deepening of voice and/or hirsutism. These reproductive characteristics of clinical hyperandrogenism may be simply diagnosed by asking the female or are apparent after a short physical examination of the female's body. Usually, a reference population does not show any or not more than one of these phenotypical characteristics known to be indicative of PCOS.

Fibroblast growth factor binding protein 1 (FGFBP1, alias FGF-BP, FGFBP1, FGFBP, FGFBP-1 or HBP17) is a 26.2 kDa protein belonging to the fibroblast growth factor binding protein family. FGFBP1 binds FGF1, FGF2, FGF7, FGF10, FGF22 and HSPG2 (perlecan) in a reversible non-covalent manner (Wu D Q, Kan M K, Sato G H, et al. Characterization and molecular cloning of a putative binding protein for heparin-binding growth factors. J Biol Chem. 1991 Sep. 5; 266(25):16778-85; Beer H D, Bittner M, Niklaus G, et al. The fibroblast growth factor binding protein is a novel interaction partner of FGF-7, FGF-10 and FGF-22 and regulates FGF activity: implications for epithelial repair. Oncogene. 2005 Aug. 11; 24(34):5269-77; Abuharbeid S, Czubayko F, Aigner A. The fibroblast growth factor-binding protein FGF-BP. Int J Biochem Cell Biol. 2006; 38 (9): 1463-8). Many members of the FGF family are immobilized in the extracellular matrix (ECM), bound to heparin sulfate proteoglycans (HSPGs) and released from this storage site by proteases and heparanases. FGFBPs shuttle FGFs from their storage sites to their receptors (Turner N, Grose R. Fibroblast growth factor signalling: from development to cancer. Nat Rev Cancer 2010, 10:116-129). Biochemical studies revealed that FGFBP1 binds to FGF2 in a dose-dependent and specific manner and that this binding is inhibited by FGF1, heparansulphate and heparinoids (Tassi E. Al-Attar A, Aigner A, et al. Enhancement of fibroblast growth factor (FGF) activity by an FGF-binding protein. J Biol Chem 2001, 276:40247-40253). This FGFBP1/FGF2 interaction results in a marked reduction of the affinity of FGF2 to heparin, and thus in the FGFBP1-mediated release of FGF2 from the ECM (Aigner A, Butscheid M, Kunkel P, et al. An FGF-binding protein (FGF-BP) exerts its biological function by parallel paracrine stimulation of tumor cell and endothelial cell proliferation through FGF-2 release. Int J Cancer 2001, 92:510-517). Several findings from different laboratories indicate that FGFBP1 can contribute to embryonic development, angiogenesis, wound healing, tumor growth, and malignant progression as well as to the maintenance and reinnervation of the neuromuscular junction and blood-brain barrier development (Czubayko F, Smith R V, Chung H C, Wellstein A. Tumor growth and angiogenesis induced by a secreted binding protein for fibroblast growth factors. J Biol Chem 1994, 269:28243-28248; Mongiat M, Otto J, Oldershaw R, et al. Fibroblast growth factor-binding protein is a novel partner for perlecan protein core. J Biol Chem 2001, 276:10263-10271; Tassi E, Al-Attar A, Aigner A, et al. Enhancement of fibroblast growth factor (FGF) activity by an FGF-binding protein. J Biol Chem 2001, 276:40247-40253; Gibby K A, McDonnell K, Schmidt M O, Wellstein A. A distinct role for secreted fibroblast growth factor-binding proteins in development, Proc Natl Acad Sci USA: 2009, 106:8585-8590; Czubayko F, Liaudet-Coopman E D, Aigner A, et al. A secreted FGF-binding protein can serve as the angiogenic switch in human cancer. Nat Med 1997, 3:1137-1140; Kurtz A, Aigner A, Cabal-Manzano R H, et al. Differential regulation of a fibroblast growth factor-binding protein during skin carcinogenesis and wound healing. Neoplasia 2004, 6:595-602; Kurtz A, Wang H L, Darwiche N, et al. Expression of a binding protein for FGF is associated with epithelial development and skin carcinogenesis. Oncogene 1997, 14:2671-2681; Tassi E, Henke R T, Bowden E T, et al. Expression of a fibroblast growth factor-binding protein during the development of adenocarcinoma of the pancreas and colon. Cancer Res 2006, 66:1191-1198; Tassi E, Wellstein A. The angiogenic switch molecule, secreted FGFbinding protein, an indicator of early stages of pancreatic and colorectal adenocarcinoma. Semin Oncol 2006, 33:S50-56; Williams A H, Valdez G, Moresi V, et al. MicroRNA-206 delays ALS progression and promotes regeneration of neuromuscular synapses in mice. Science 2009, 326:1549-1554; Tassi E, McDonnell K, Gibby K A, et al. Impact of fibroblast growth factor-binding protein-1 expression on angiogenesis and wound healing. Am J Pathol. 2011 November; 179(5):2220-32; Cottarelli A, Corada M, Beznoussenko G V, et al. Fgfbpl promotes blood-brain barrier development by regulating collagen IV deposition and maintaining Wnt/β-catenin signaling. Development. 2020 Aug. 24; 147(16):dev185140). FGFBP1 mRNA is expressed in mouse normal skin, lung, intestine, ovaries, placenta, stomach and eye (Fon Tacer K, Bookout A L, Ding X, et al. Research resource: comprehensive expression atlas of the Fibroblast Growth Factor system in adult mouse. Mol Endocrinol 2010, 24:2050-2064; Kurtz A, Wang H L, Darwiche N. et al. Expression of a binding protein for FGF is associated with epithelial development and skin carcinogenesis. Oncogene 1997, 14:2671-2681). A genetic polymorphism in the human FGFBP1 gene was associated with higher gene and protein expression in the human kidney and an increased risk of familial hypertension (Tomaszewski M, Charchar F J, Nelson C P, et al. Pathway analysis shows association between FGFBP1 and hypertension. J Am Soc Nephrol. 2011 May; 22(5):947-55). It has been shown that modulation of FGF signaling by FGFBP1 can regulate vascular sensitivity to endogenous angiotensin II and hence control steady-state blood pressure (Tassi E, Lai E Y, Li L, et al. Blood Pressure Control by a Secreted FGFBP1 (Fibroblast Growth Factor-Binding Protein). Hypertension. 2018 January; 71(1):160-167). FGF2 signaling has proven to be central for the maintenance of cellular plasticity of epithelial ovarian cancer already in the very initial steps of carcinogenesis (De Cecco L, Marchionni L, Gariboldi M, et al. Gene expression profiling of advanced ovarian cancer: characterization of a molecular signature involving fibroblast growth factor 2. Oncogene. 2004 Oct. 21; 23 (49): 8171-83). Higher levels of serum FGF2 have been found in patients with epithelial ovarian cancer (both in benign and malignant subtypes) compared to healthy controls (Barton D P, Cai A. Wendt K, et al. Angiogenic protein expression in advanced epithelial ovarian cancer. Clin Cancer Rcs 3:1579-1586, 1997; Le Page C, Ouellet V, Madore J, et al. From gene profiling to diagnostic markers: IL-18 and FGF-2 complement CA125 as serum-based markers in epithelial ovarian cancer. Int J Cancer 118:1750-1758, 2006; Madsen C V, Steffensen K D, Olsen D A, et al. Serum platelet-derived growth factor and fibroblast growth factor in patients with benign and malignant ovarian tumors. Anticancer Res. 2012 September; 32(9):3817-25). In rat, FGF2 treatment of ovaries cultures inhibited primordial follicle assembly. Furthermore, a number of differentially expressed genes identified in the treated ovaries correlated to previously known genes linked to PCOS, suggesting that FGF2-dependent abnormal follicle assembly could be a component for PCOS later in life (Nilsson E, Zhang B, Skinner M K. Gene bionetworks that regulate ovarian primordial follicle assembly. BMC Genomics. 2013 Jul. 23; 14:496). In cows, FGF2 mRNA expression is high in preovulatory follicles and in the early luteal phase, it then decreases in the late luteal phase and remains at low levels during pregnancy (Berisha B, Schams D, Rodler D, Pfaffl M W. Angiogenesis in The Ovary—The Most Important Regulatory Event for Follicle and Corpus Luteum Development and Function in Cow—An Overview. Anat Histol Embryol. 2016 April; 45(2):124-30). Intraperitoneal injection of FGF2, in a mouse model of estradiol valerate induced PCOS, had protective and ameliorative effects (Moayeri A, Rostamzadeh A, Raoofi A, et al. Retinoic acid and fibroblast growth factor-2 play a key role on modulation of sex hormones and apoptosis in a mouse model of polycystic ovary syndrome induced by estradiol valerate. Taiwan J Obstet Gynecol. 2020 November; 59(6):882-890). Controversial results about serum FGF2 levels in PCOS patients have been published. Artini and colleagues reported no differences in scrum FGF2 levels between untreated PCOS and control patients, but increased levels of FGF2 in PCOS patients after FSH stimulation (Artini P G, Monti M, Matteucci C, et al. Vascular endothelial growth factor and basic fibroblast growth factor in polycystic ovary syndrome during controlled ovarian hyperstimulation. Gynecol Endocrinol. 2006 August; 22(8):465-70). Patil and colleagues instead reported lower serum FGF2 levels in PCOS women undergoing controlled ovarian hyperstimulation for IVF compared to age and BMI matched controls (Patil K, Hinduja I, Mukherjee S. Alteration in angiogenic potential of granulosa-lutein cells and follicular fluid contributes to luteal defects in polycystic ovary syndrome. Hum Reprod. 2021 Mar. 18; 36(4):1052-1064). While the role of FGF2-signaling is unclear in PCOS, its metabolic function has been investigated in recent years. FGF2, depending on concentration, can function as either a positive or a negative factor of in vitro adipogenesis (Kim S, Ahn C, Bong N, et al. Biphasic effects of FGF2 on adipogenesis. PLOS One. 2015 Mar. 19; 10(3):e0120073. doi: 10.1371/journal.pone.0120073). Mathes and colleagues showed that FGF2-dependent signaling enhances the differentiation of fibro/adipogenic progenitors promoting the formation of intramuscular adipose tissue (Mathes S, Fahrner A, Ghoshdastider U, et al. FGF-2-dependent signaling activated in aged human skeletal muscle promotes intramuscular adipogenesis. Proc Natl Acad Sci USA. 2021 Sep. 14; 118 (37): e2021013118. doi: 10.1073/pnas.2021013118). The appearance of adipose tissue between skeletal muscle fibers is a unique feature of aging, obesity and type 2 diabetes, demonstrating association with insulin resistance. Furthermore, FGF2 has been shown to be a negative regulator of thermogenesis in both brown and beige fat. Disruption of FGF2 strongly enhanced the thermogenic action of brown and beige fat, leading to increased energy expenditure and improved lipid homeostasis. In addition, deletion of FGF2 protected mice from adiposity and hepatic steatosis induced by high fat levels. (Li H, Zhang X, Huang C, et al. FGF2 disruption enhances thermogenesis in brown and beige fat to protect against adiposity and hepatic steatosis. Mol Metab. 2021 December; 54:101358. doi: 10.1016/j.molmet.2021).

The first aspect of the present invention relates to a method of assessing whether a subject has PCOS or is at risk of developing PCOS comprising;
(a) Determining the amount or concentration of FGFBP1 in a sample of the subject, and
(b) Comparing the determined amount or concentration to a reference.

An elevated amount or concentration of FGFBP1 in the sample of the patient is indicative of the presence or the risk or developing PCOS in the patient. In particular, an amount or concentration of FGFBP1 in the sample of the patient is indicative of the presence or the risk of developing PCOS in the patient if the amount or concentration of FGFBP1 in the sample of the patient is higher than the amount or concentration of FGFBP1 in a reference or a reference sample. In particular, FGFBP1 is detectable in higher amounts or concentrations in a biological fluid sample of the patient assessed for the presence or risk of developing PCOS than in the same biological fluid sample of individuals not suffering or being at risk of developing PCOS. In particular an amount or concentration of FGFBP1 elevated by 50% or more, is indicative of the presence or risk of developing PCOS. In particular an amount or concentration of FGFBP1 elevated by 100% or more, is indicative of the presence or risk of developing PCOS. In particular an amount or concentration of FGFBP1 elevated by 150% or more, is indicative of the presence or risk of developing PCOS. In particular an amount or concentration of FGFBP1 elevated by 200% or more, is indicative of the presence or risk of developing PCOS.

In embodiments, the biological fluid sample is whole blood, serum, plasma, capillary blood, interstitial fluid, peritoneal fluid, or menstrual fluid preferably the biological fluid sample is serum or whole blood. In embodiments, the sample is an in vitro sample, i.e. it will be analyzed in vitro and not transferred back to the body.

In particular embodiments, the patient is a laboratory animal, a domestic animal or a primate. In particular embodiments, the patient is a human patient. In particular embodiments, the patient is a female human patient. In particular embodiments, the patient is a female human patient of less than 25 years old. In particular embodiments, the patient is a female human patient of less than 20 years old. In particular embodiments, the patient is a female human patient of 15 to less than 25 years old. In particular embodiments, the patient is a female human patient of 15 to less than 20 years old. In particular embodiments, the patient is a female human patient of less than 25 years old and three years after menarche. In particular embodiments, the patient is a female human patient of less than 20 years old and three years after menarche. In particular embodiments, the patient is a female human patient of 15 to less than 25 years old and three years after menarche. In particular embodiments, the patient is a female human patient of 15 to less than 20 years old and three years after menarche.

In embodiments, PCOS is assessed from the group consisting of metabolic or phenotypical PCOS. In further embodiments, PCOS is assessed from the group consisting of phenotype A, phenotype B, phenotype C and phenotype D PCOS.

In embodiments, the first method of the present invention is an in vitro method.

In embodiments, the amount or concentration of FGFBP1 is determined using antibodies, in particular using monoclonal antibodies. In embodiments, step a) of determining the amount or concentration of FGFBP1 in a sample of the patient comprises performing an immunoassay. In embodiments, the immunoassay is performed either in a direct or indirect format. In embodiments, such immunoassay is selected from the group consisting of enzyme linked immunosorbent assay (ELISA), enzyme immunoassay (EIA), radioimmunoassay (RIA), or immuno assay based on detection of luminescence, fluorescence, chemiluminescence or electrochemiluminescence.

In particular embodiments, step a) of determining the amount or concentration of FGFBP1 in a sample of the patient comprises the steps of:
i) incubating the sample of the patient with one or more antibodies specifically binding to FGFBP1, thereby generating a complex between the antibody and FGFBP1, and
ii) quantifying the complex formed in step i), thereby quantifying the amount or concentration of FGFBP1 in the sample of the patient.

In particular embodiments, in step i) the sample is incubated with two antibodies, specifically binding to FGFBP1. As obvious to the person skilled in the art, the sample can be contacted with the first and the second antibody in any desired order, e.g. First antibody first and then the second antibody or second antibody first and then the first antibody, or simultaneously, for a time and under conditions sufficient to form a first anti-FGFBP1 antibody/FGFBP1/second anti-FGFBP1 antibody complex. As the person skilled in the art will readily appreciate it is nothing but routine experimentation to establish the time and conditions that are appropriate or that are sufficient for the formation of a complex either between the specific anti-FGFBP1 antibody and the FGFBP1 antigen/analyte (=anti-FGFBP1 complex) or the formation of the secondary, or sandwich complex comprising the first antibody to FGFBP1, FGFBP1 (the analyte) and the second anti-FGFBP1 antibody (=first anti-FGFBP1 antibody/FGFBP1/second anti-FGFBP1 antibody complex).

The detection of the anti-FGFBP1 antibody/FGFBP1 complex can be performed by any appropriate means. The detection of the first anti-FGFBP1 antibody/FGFBP1/second anti-FGFBP1 antibody complex can be performed by any appropriate means. The person skilled in the art is absolutely familiar with such means/methods.

In certain embodiments, a sandwich will be formed comprising a first antibody to FGFBP1, FGFBP1 (analyte) and the second antibody to FGFBP1, wherein the second antibody is detectably labeled.

In one embodiment, a sandwich will be formed comprising a first antibody to FGFBP1, FGFBP1 (analyte) and the second antibody to FGFBP1, wherein the second antibody is detectably labeled and wherein the first anti-FGFBP1 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In a second aspect, the present invention relates to a method of selecting a patient for therapy of PCOS, comprising:
(c) Determining the amount or concentration of FGFBP1 in a sample of the subject, and
(d) Comparing the determined amount or concentration to a reference.

In embodiments, a patient is selected for therapy of PCOS if an elevated amount of FGFBP1 in the sample of the patient is determined. In particular, a patient is selected for therapy of PCOS if the amount of FGFBP1 is higher than the amount of FGFBP1 in a reference or reference sample. In particular, a patient is selected for therapy of PCOS if the amount of FGFBP1 is higher in a biological fluid sample of the patient assessed for therapy of PCOS than in the same biological fluid sample of individuals not suffering or being at risk of developing PCOS or not being selected for therapy of PCOS. In particular a patient is selected for therapy of PCOS if the amount of FGFBP1 is elevated by 50% or more. In particular, a patient is selected for therapy of PCOS if the amount of FGFBP1 is elevated by 100% or more. In particular, a patient is selected for therapy of PCOS if the amount of FGFBP1 is elevated by 150% or more. In particular, a patient is selected for therapy of PCOS if the amount of FGFBP1 is elevated by 200% or more.

In particular, the patient is selected for a drug-based therapy of PCOS or for lifestyle changes to control metabolic symptoms. In embodiments, drug-based therapy of PCOS is selected from the group consisting of drugs for regulating periods, in particular oral contraceptives or progestin therapy, drugs for preventing or controlling diabetes, in particular type 2 diabetes, drugs for preventing or controlling high cholesterol, hormones or drugs to increase fertility, drugs, hormones or procedures to remove excess hair, drugs or procedure to control acne.

In embodiments, the biological fluid sample is whole blood, serum, plasma, capillary blood, interstitial fluid, peritoneal fluid, or menstrual fluid preferably the biological fluid sample is serum or whole blood. In embodiments, the sample is an in vitro sample, i.e. it will be analyzed in vitro and not transferred back to the body.

In particular embodiments, the patient is a laboratory animal, a domestic animal or a primate. In particular embodiments, the patient is a human patient. In particular embodiments, the patient is a female human patient. In particular embodiments, the patient is a female human patient of less than 25 years old. In particular embodiments, the patient is a female human patient of less than 20 years old. In particular embodiments, the patient is a female human patient of 15 to less than 25 years old. In particular embodiments, the patient is a female human patient of 15 to less than 20 years old. In particular embodiments, the patient is a female human patient of less than 25 years old and three years after menarche. In particular embodiments, the patient is a female human patient of less than 20 years old and three years after menarche. In particular embodiments, the patient is a female human patient of 15 to less than 25 years old and three years after menarche. In particular embodiments, the patient is a female human patient of 15 to less than 20 years old and three years after menarche.

In embodiments, the second method of the present invention is an in vitro method.

In embodiments, the amount of FGFBP1 is determined using antibodies, in particular using monoclonal antibodies. In embodiments, step a) of determining the amount of FGFBP1 in a sample of the patient comprises performing an immunoassay. In embodiments, the immunoassay is performed either in a direct or indirect format. In embodiments, such immunoassay is selected from the group consisting of enzyme linked immunosorbent assay (ELISA), enzyme e immunoassay (EIA), radioimmunoassay (RIA), or immuno assay based on detection of luminescence, fluorescence, chemiluminescence or electrochemiluminescence.

In particular embodiments, step a) of determining the amount of FGFBP1 in a sample of the patient comprises the steps of:
i) incubating the sample of the patient with one or more antibodies specifically binding to FGFBP1, thereby generating a complex between the antibody and FGFBP1, and
ii) quantifying the complex formed in step i), thereby quantifying the amount of FGFBP1 in the sample of the patient.

In particular embodiments, in step i) the sample is incubated with two antibodies, specifically binding to FGFBP1. As obvious to the person skilled in the art, the sample can be contacted with the first and the second antibody in any desired order, i.g. First antibody first and then the second antibody or second antibody first and then the first antibody, or simultaneously, for a time and under conditions sufficient to form a first anti-FGFBP1 antibody/FGFBP1/second anti-FGFBP1 antibody complex. As the person skilled in the art will readily appreciate it is nothing bu routine experimentation to establish the time and conditions that are appropriate or that are sufficient for the formation of a complex either between the specific anti-FGFBP1 antibody and the FGFBP1 antigen/analyte (=anti-FGFBP1 complex) or the formation of the secondary, or sandwich complex comprising the first antibody to FGFBP1, FGFBP1 (the analyte) and the second anti-FGFBP1 antibody (=first anti-FGFBP1 antibody/FGFBP1/second anti-FGFBP1 antibody complex).

The detection of the anti-FGFBP1 antibody/FGFBP1 complex can be performed by any appropriate means. The detection of the first anti-FGFBP1 antibody/FGFBP1/second anti-FGFBP1 antibody complex can be performed by any appropriate means. The person skilled in the art is absolutely familiar with such means/methods.

In certain embodiments, a sandwich will be formed comprising a first antibody to FGFBP1, FGFBP1 (analyte) and the second antibody to FGFBP1, wherein the second antibody is detectably labeled.

In one embodiment, a sandwich will be formed comprising a first antibody to FGFBP1, FGFBP1 (analyte) and the second antibody to FGFBP1, wherein the second antibody is detectably labeled and wherein the first anti-FGFBP1 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

Further, the present invention relates also to a kit comprising reagents for the diagnosis of PCOS. The reagents of the kit may comprise antibodies or antibody fragments. Preferably, the antibodies or antibody fragments recognize epitopes or antigens of FGFBP1. The kit may further contain other reagents which recognize other biomarkers. Therefore, the kit may comprise a combination of at least two reagents. The kit may specifically measure the amount or concentration of FGFBP1 and any other biomarker of interest. According to the present invention, a biomarker can also comprise hormones, such as Anti-Müllerian Hormone, AMH. The kit can be used in any diagnostic assay.

In embodiments, the amount of FGFBP1 is determined using antibodies, in particular using monoclonal antibodies. In embodiments, step a) of determining the amount of FGFBP1 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In a third aspect, the present invention relates to a method for monitoring PCOS progression in a subject or for monitoring response to treatment in a subject having PCOS, said method comprising:
(a) determining the level of FGFBP1 in a first sample of the subject,
(b) determining the level of FGFBP1 in a second sample of the subject which has been obtained after the first sample, and
(c) comparing the level of FGFBP1 in the first sample to the level of FGFBP1 in the second sample, and
(d) monitoring progression in the subject suffering from PCOS or being treated for PCOS, based on the results of step c).

In embodiments, PCOS progression in a subject having PCOS is monitored to determine if the amount or concentration of FGFBP1 is changing over time in a sample of the patient. In particular, PCOS progression is monitored to determine if the amount or concentration of FGFBP1 is increasing, decreasing or not changing over time. In embodiments, PCOS progression is monitored if an elevated amount or concentration of FGFBP1 in the sample of the subject is determined.

In embodiments, a subject being treated for PCOS is monitored to determine if the amount or concentration of FGFBP1 is changing in a sample of the subject. In particular, a subject being treated for PCOS is monitored to determine if the amount or concentration of FGFBP1 is increasing, decreasing or not changing. In particular, a subject being treated for PCOS is monitored to determine if the amount or concentration of FGFBP1 is increasing, decreasing or not changing due to the therapy applied. In embodiments, a decreasing amount or concentration of FGFBP1 in a subject being treated for PCOS is indicative of the therapy being effective. In embodiments, an unaltered or increasing amount or concentration of FGFBP1 in a sample of the subject being treated for PCOS is indicative of persisting PCOS. In particular, the treatment for PCOS is ineffective if the amount or concentration of FGFBP1 is increasing to 50% or more. In particular, the treatment for PCOS is ineffective if the amount or concentration of FGFBP1 is increasing to 100% or more. In particular, the treatment for PCOS is ineffective if the amount or concentration of FGFBP1 is increasing to 150% or more. In particular, the treatment for PCOS is ineffective if the amount or concentration of FGFBP1 is increasing to 200% or more.

In particular embodiments, therapy is adapted if an unaltered or increasing amount or concentration of FGFBP1 in a sample of the subject being treated for PCOS is determined.

In embodiments, the subject is monitored several times at different time points. In embodiments, the subject is monitored several times within a time frame of weeks, months or years. In particular embodiments, a subject is monitored once a month or once a year. In embodiments, a subject suffering from PCOS is monitored once a month or once a year after diagnosis of PCOS. In embodiments, a subject being treated for PCOS is monitored once after therapy. In particular, the subject being treated for PCOS is monitored once a month or once a year to determine the efficacy of treatment.

In embodiments, therapy of PCOS is selected from the group consisting of drug-based therapy of PCOS and lifestyle changes to control metabolic symptoms. In embodiments, drug-based therapy of PCOS is selected from the group consisting of drugs for regulating periods, in particular oral contraceptives or progestin therapy, drugs for preventing or controlling diabetes, in particular type 2 diabetes, drugs for preventing or controlling high cholesterol, hormones or drugs to increase fertility, drugs, hormones or procedures to remove excess hair, drugs or procedure to control acne.

In embodiments, the biological fluid sample is whole blood, serum, plasma, capillary blood, interstitial fluid, peritoneal fluid, or menstrual fluid preferably the biological fluid sample is serum or whole blood. In embodiments, the sample is an in vitro sample, i.e. it will be analyzed in vitro and not transferred back to the body.

In particular embodiments, the patient is a laboratory animal, a domestic animal or a primate. In particular embodiments, the patient is a human patient. In particular embodiments, the patient is a female human patient. In particular embodiments, the patient is a female human patient of less than 25 years old. In particular embodiments, the patient is a female human patient of less than 20 years old. In particular embodiments, the patient is a female human patient of 15 to less than 25 years old. In particular embodiments, the patient is a female human patient of 15 to less than 20 years old. In particular embodiments, the patient is a female human patient of less than 25 years old and three years after menarche. In particular embodiments, the patient is a female human patient of less than 20 years old and three years after menarche. In particular embodiments, the patient is a female human patient of 15 to less than 25 years old and three years after menarche. In particular embodiments, the patient is a female human patient of 15 to less than 20 years old and three years after menarche.

In embodiments, the second method of the present invention is an in vitro method.

In embodiments, the amount or concentration of FGFBP1 is determined using antibodies, in particular using monoclonal antibodies. In embodiments, step a) of determining the amount or concentration of FGFBP1 in a sample of the patient comprises performing an immunoassay. In embodiments, the immunoassay is performed either in a direct or indirect format. In embodiments, such immunoassay is selected from the group consisting of enzyme linked immunosorbent assay (ELISA), enzyme immunoassay (EIA), radioimmunoassay (RIA), or immuno assay based on detection of luminescence, fluorescence, chemiluminescence or electrochemiluminescence.

In particular embodiments, step a) of determining the amount or concentration of FGFBP1 in a sample of the patient comprises the steps of:
i) incubating the sample of the patient with one or more antibodies specifically binding to FGFBP1, thereby generating a complex between the antibody and FGFBP1, and
ii) quantifying the complex formed in step i), thereby quantifying the amount or concentration of FGFBP1 in the sample of the patient.

In particular embodiments, in step i) the sample is incubated with two antibodies, specifically binding to FGFBP1. As obvious to the person skilled in the art, the sample can be contacted with the first and the second antibody in any desired order, e.g. first antibody first and then the second antibody or second antibody first and then the first antibody, or simultaneously, for a time and under conditions sufficient to form a first anti-FGFBP1 antibody/FGFBP1/second anti-FGFBP1 antibody complex. As the person skilled in the art will readily appreciate it is nothing but routine experimentation to establish the time and conditions that are appropriate or that are sufficient for the formation of a complex either between the specific anti-FGFBP1 antibody and the FGFBP1 antigen/analyte (=anti-FGFBP1 complex) or the formation of the secondary, or sandwich complex comprising the first antibody to FGFBP1, FGFBP1 (the analyte) and the second anti-FGFBP1 antibody (=first anti-FGFBP1 antibody/FGFBP1/second anti-FGFBP1 antibody complex).

The detection of the anti-FGFBP1 antibody/FGFBP1 complex can be performed by any appropriate means. The detection of the first anti-FGFBP1 antibody/FGFBP1/second anti-FGFBP1 antibody complex can be performed by any appropriate means. The person skilled in the art is absolutely familiar with such means/methods.

In certain embodiments, a sandwich will be formed comprising a first antibody to FGFBP1, FGFBP1 (analyte) and the second antibody to FGFBP1, wherein the second antibody is detectably labeled.

In one embodiment, a sandwich will be formed comprising a first antibody to FGFBP1, FGFBP1 (analyte) and the second antibody to FGFBP1, wherein the second antibody is detectably labeled and wherein the first anti-FGFBP1 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In a fourth aspect, the present invention relates to a computer-implemented method of assessing a subject with suspected PCOS comprising the steps of:
(a) receiving a value for the amount or concentration of a first biomarker in a sample of the subject, said first biomarker being FGFBP1,
(b) optionally, receiving a value for the amount or concentration of a second biomarker in a sample of the subject,
(c) optionally, receiving a value for the presence or absence of at least an additional diagnostic criterion selected from the group consisting of oligo-anovulation, hyperandrogenism and polycystic ovarian morphology;
(d) comparing the values for the amounts or concentrations of steps (a)-(b) to references for said biomarkers and the value for the presence or absence of the at least one additional diagnostic criterion, and/or calculating a score for assessing the subject with suspected PCOS based on the amounts or concentrations of the biomarkers and the value; and
(e) assessing said subject based on the comparison and/or the calculation made in step (d).

In embodiments, the computer-implemented method of assessing a subject with suspected PCOS includes methods which essentially consist of the aforementioned steps or methods which include further steps. Moreover, the method of the present invention, preferably, is an ex vivo and more preferably an in vitro method. Moreover, it may comprise steps in addition to those explicitly mentioned above. For example, further steps may relate to the determination of further markers and/or to sample pre-treatments or evaluation of the results obtained by the method. The method may be carried out manually or assisted by automation.

The term “computer-implemented” as used herein means that the method is carried out in an automated fashion on a data processing unit which is, typically, comprised in a computer or similar data processing device. The data processing unit shall receive values for the amount of the biomarkers. Such values can be the amounts, relative amounts or any other calculated value reflecting the amount as described elsewhere herein in detail. Accordingly, it is to be understood that the aforementioned method does not require the determination of amounts for the biomarkers but rather uses values for already predetermined amounts.

The present invention also, in principle, contemplates a computer program, computer program product or computer readable storage medium having tangibly embedded said computer program, wherein the computer program comprises instructions which, when run on a data processing device or computer, carry out the method of the present invention as specified above. Specifically, the present disclosure further encompasses:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a data for parameters as defined herein elsewhere, and
a data stream signal, typically encrypted, comprising the assessment provided by the methods of the present invention.

### Definitions

In the context of the kit of the present invention the term “reagent” describes a substance or compound added to a sample allowing to display the amount or concentration of a specific component in the sample.

In the context of the kit of the present invention the term “specifically measure” means to detect the exact amount or concentration of a clearly defined molecule. For a specific measurement the sample obtained from a female may be incubated with the reagent under conditions appropriate for formation of a binding agent marker-complex. Such conditions need not be specified, since such appropriate incubation conditions are well-known to the skilled artisan.

In the context of the kit of the present invention the term “reagent” may describe a protein molecule (such as an antibody), a nucleic acid molecule (such as any form of deoxyribonucleic acid (DNA) or ribonucleic acid (RNA)) or another biochemical, organic or anorganic substance, which may interact with the molecule to be specifically measured in a sample.

Further, the reagent may be linked to a detectable reporter moiety or label such as an enzyme, dye, radionuclide, luminescent group, fluorescent group or biotin, or the like, such as a fluorescent marker that may be used for immunoassays analysis. Any reporter moiety or label could be used with the reagent of the kit according to the second aspect of the invention so long as the signal of such may be directly related or proportional to the quantity of binding agent remaining on the support after wash. The amount of an optional second binding agent that remains bound to the solid support may then be determined using a method appropriate for the specific detectable reporter moiety or label. For radioactive groups, scintillation counting or autoradiographic methods are generally appropriate. Antibody-enzyme conjugates can be prepared using a variety of coupling techniques (for review see, e.g., Scouten, W. H., Methods in Enzymology 135:30-65, 1987). Spectroscopic methods can be used to detect dyes (including, for example, colorimetric products of enzyme reactions), luminescent groups and fluorescent groups. Biotin can be detected using avidin or streptavidin, coupled to a different reporter group (commonly a radioactive or fluorescent group or an enzyme). Enzyme reporter groups can generally be detected by the addition of substrate (generally for a specific period of time), followed by spectroscopic, spectrophotometric or other analysis of the reaction products. Standards and standard additions can be used to determine the level of antigen in a sample, using techniques well-known to the person skilled in the art.

The reagent may also be a substance that may additionally be capable of linking to the matrix of a column used for chromatography for purification and/or further analysis (such as mass spectrometry analysis). Moreover, the reagent may be linked to a testing strip.

Preferably, the reagent is an antibody. Suitable antibodies for measuring the amount or concentration of one of the molecules to be specifically measured in a sample obtained from the female mentioned above are well-known to the person skilled in the art.

Preferably, the reagent may be used in an electrochemiluminescence-immunoassay, more preferably, the reagent is an antibody that may be used in an electrochemiluminescence-immunoassay.

Moreover, the kit may comprise more than one reagent, such as two different reagents, three different reagents, four different reagents or more different reagents, preferably two different reagents to interact with one molecule that is specifically measured in a sample. For example, if the molecule that is specifically measured is measured in an electrochemiluminescence-immunoassay, the kit may comprise two different antibodies binding to the same molecule to be measured. Preferably, the two different antibodies binding to the same molecule are not competing for the binding site at the molecule and bind this molecule at different positions. Further, both antibodies may be linked to different detectable reporter moieties or labels.

The kit may further comprise buffering agents and/or salts to adjust the pH as well as the reaction and measuring conditions. Moreover, the kit may comprise stabilizers, e.g. to support the stability of the reagents and/or hormones during the specific measurement of (i) the amount or concentration of FT or (ii) the amount or concentration of TT and the amount or concentration SHBG; the amount or concentration of AMH; and the amount or concentration of one or more further hormones indicative of PCOS. Suitable buffers, salts and stabilizers are well-known to the person skilled in the art. In addition, sodium azide may be added to all liquid solutions of the kit, such as reagents or buffers.

The kit may also comprise all equipment necessary to take a blood sample from a female, such as a container for the blood sample, a needle and a device connecting the container and the needle. Preferably, the kit may comprise a syringe.

In general, a physician or a physician's assistant may take blood from a female. Subsequently, the blood may be sent to a laboratory, where the sample is measured using the kit on a designated analyser, and the data are sent to the physician. However, the kit may also be applied by a physician or by a physician's assistant himself. The kit may be applied during ambulatory, stationary treatment or domiciliary visit of the physician.

All components of the kit may be packaged separately in individual containers. However, it is also possible that two or more components of the kit may be packaged together in one or more containers.

The kit may further comprise a label, e.g. comprising instructions on how to use the kit or describing the kit's contents. However, this information may also be provided in any other form, such as on storage media such as a CD-ROM or a USB stick.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “indicator” as used herein, refers to a sign or signal for a condition or is used to monitor a condition. Such a “condition” refers to the biological status of a cell, tissue or organ or to the health and/or disease status of an individual. An indicator may be the presence or absence of a molecule, including but not limited to peptide, protein, and nucleic acid, or may be a change in the expression level or pattern of such molecule in a cell, or tissue, organ or individual. An indicator may be a sign for the onset, development or presence of a disease in an individual or for the further progression of such disease. An indicator may also be a sign for the risk of developing a disease in an individual.

In the context of present invention, the term “biomarker” refers to a substance within a biological system that is used as an indicator of a biological state of said system. In the art, the term, “biomarker” is sometimes also applied to means for the detection of said endogenous substances (e.g. antibodies, nucleic acid probes etc, imaging systems). In the context of present invention, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA) or a substance that has been internalized by the organism or a metabolite of such a substance.

The biomarkers as referred to herein can be detected using methods generally known in the art. Methods of detection generally encompass methods to quantify the level of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays and proximity extension assays, which are commercially available. Further suitable methods to detect biomarkers include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

The term “anovulation” usually describes the condition when the ovaries do not release any oocyte during a female menstrual cycle at all. The female whose risk of having PCOS is assessed may be determined to suffer from anovulation, if no oocyte is released for the duration of at least one female menstrual cycle, preferably at least three female menstrual cycles, more preferably at least six female menstrual cycles and mostly preferred at least nine female menstrual cycles in one year. Further, the female whose risk of having PCOS is assessed may be determined to suffer from anovulation, if no oocyte is released for the duration of at least 6 months, preferably at least 9 months, more preferably at least 1 year.

“Symptoms” of a disease are implication of the disease noticeable by the tissue, organ or organism having such disease and include but are not limited to pain, weakness, tenderness, strain, stiffness, and spasm of the tissue, an organ or an individual. Symptoms typical for PCOS include but are not limited to oligo-anovulation, irregular cycles, hyperandrogenism, polycystic ovarian morphology, infertility, diabetes type 2, overweight and other metabolic conditions, and psychological distress. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfill its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterized by an increase or decrease of such symptoms or signs which may indicate a “worsening” or “bettering” of the disease. The “worsening” of a disease is characterized by a decreasing ability of a tissue, organ or organism to fulfill its function efficiently, whereas the “bettering” of a disease is typically characterized by an increase in the ability of a tissue, an organ or an individual to fulfill its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to inflammatory diseases, infectious diseases, cutaneous conditions, endocrine diseases, intestinal diseases, neurological disorders, joint diseases, genetic disorders, autoimmune diseases, traumatic diseases, and various types of cancer.

The terms “patient” and “subject” are used interchangeably herein, referring to an animal, preferably a mammal and, more typically to a human. The patient is preferably a human female. There is a need for diagnosis of PCOS.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, it will be analyzed in vitro and not transferred back into the body.

The term “amount” as used herein encompasses the absolute amount of a biomarker as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts measured from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the measured or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provides the desired assessment in a suitable output format.

The expression “comparing the amount or concentration determined to a reference” is merely used to further illustrate what is obvious to the skilled artisan anyway. A reference concentration is established in a control sample

The term “reference sample” or “control sample” as used herein, refers to a sample which is analysed in a substantially identical manner as the sample of interest and whose information is compared to that of the sample of interest. A reference sample thereby provides a standard allowing for the evaluation of the information obtained from the sample of interest. A control sample may be derived from a healthy or normal tissue, organ or individual, thereby providing a standard of a healthy status of a tissue, organ or individual. Differences between the status of the normal reference sample and the status of the sample of interest may be indicative of the risk of disease development or the presence or further progression of such disease or disorder. A control sample may be derived from an abnormal or diseased tissue, organ or individual thereby providing a standard of a diseased status of a tissue, organ or individual. Differences between the status of the abnormal reference sample and the status of the sample of interest may be indicative of a lowered risk of disease development or the absence or bettering of such disease or disorder. A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time.

The control sample may be an internal or an external control sample. An internal control sample is used, i.e. the marker level(s) is (are) assessed in the test sample as well as in one or more other sample(s) taken from the same subject to determine if there are any changes in the level(s) of said marker(s). For an external control sample the presence or amount of a marker in a sample derived from the individual is compared to its presence or amount in an individual known to suffer from, or known to be at risk of, a given condition; or an individual known to be free of a given condition, i.e., “normal individual”.

It will be appreciated by the skilled artisan that such external control sample may be obtained from a single individual or may be obtained from a reference population that is age-matched and free of confounding diseases. Typically, samples from 100 well-characterized individuals from the appropriate reference population are used to establish a “reference value”. However, reference population may also be chosen to consist of 20, 30, 50, 200, 500 or 1000 individuals. Healthy individuals represent a preferred reference population for establishing a control value.

For example, a marker concentration in a patient sample can be compared to a concentration known to be associated with a specific course of a certain disease. Usually the sample's marker concentration is directly or indirectly correlated with a diagnosis and the marker concentration is e.g. used to determine whether an individual is at risk for a certain disease. Alternatively, the sample's marker concentration can e.g. be compared to a marker concentration known to be associated with a response to therapy in a certain disease, the diagnosis of a certain disease, the assessment of the severity of a certain disease, the guidance for selecting an appropriate drug to a certain disease, in judging the risk of disease progression, or in the follow-up of patients. Depending on the intended diagnostic use an appropriate control sample is chosen and a control or reference value for the marker established therein. As also clear to the skilled artisan, the absolute marker values established in a control sample will be dependent on the assay used.

The term “assessing” as used herein refers to assessing whether a patient suffers from PCOS or is at risk of developing PCOS. Accordingly, assessing as used herein includes diagnosing PCOS, predicting the risk for developing PCOS, selecting for therapy of PCOS, monitoring a patient suffering from PCOS or being treated for PCOS, by determining the amount or concentration of FGFBP1 in a sample of the patient, and comparing the determined amount or concentration to a reference.

As will be understood by those skilled in the art, the assessment made in accordance with the present invention, although preferred to be, may usually not be correct for 100% of the investigated subjects. The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc.. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The terms “lowered” or “decreased” level, amount and/or concentration of an indicator refer to the level, amount and/or concentration of such indicator in the sample being reduced in comparison to the reference or reference sample.

The terms “elevated” or “increased” level, amount and/or concentration of an indicator refer to the level, amount and/or concentration of such indicator in the sample being higher in comparison to the reference or reference sample. E.g., a protein that is detectable in higher amounts or concentrations in a fluid sample of one individual suffering from a given disease than in the same fluid sample of individuals not suffering from said disease, has an elevated level.

The term “measurement”, “measuring” or “determining” preferably comprises a qualitative, a semi-quantitative or a quantitative measurement.

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CH1, CH2, CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCy1-Cy2-Cy3), whereas the human IgG light chain is composed of two immunoglobulin domains linked from N- to C-terminus in the order VL-CL, being either of the kappa or lambda type (VK-CK or VA.-CA.). Exemplified, the constant chain of human IgG comprises 447 amino acids. Throughout the present specification and claims, the numbering of the amino acid positions in an immunoglobulin are that of the “EU index” as in Kabat. E. A., Wu, T. T., Perry, H. M., Gottesman, K. S., and Foeller, C., (1991) Sequences of proteins of immunological interest, 5^{th} ed. U.S. Department of Health and Human Service, National Institutes of Health, Bethesda, MD. The “EU index as in Kabat” refers to the residue numbering of the human IgG 1EU antibody. Accordingly, CH domains in the context of IgG are as follows: “CH1” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The terms “full-length antibody”, “intact antibody”, and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG. IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fah′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)₂, Fv fragments; diabodies; sdAb, nanobodies, scFv, di-scFvs, tandem scFvs, triabodies, diabodies, scDb, BiTEs, and DARTs.

The term “binding affinity” generally refers to the strength of the sum total of noncovalent interactions between a single binding site of a molecule (e.g., an antibody) and its binding partner (e.g., an antigen). Unless indicated otherwise, as used herein. “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g. an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g. paramagnetic streptavidin-coated microparticles. Such microparticles are mixed and incubated with an analyte-specific binding agent bound to the second partner of the binding pair (e.g. a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s).

The term “detectably labeled” encompasses labels that can be directly or indirectly detected.

Directly detectable labels either provide a detectable signal or they interact with a second label to modify the detectable signal provided by the first or second label, e.g. to give FRET (fluorescence resonance energy transfer). Labels such as fluorescent and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e. to a fluorescent label, to a luminescent label (e.g. a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

### (a) Fluorescent Dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as clectrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+ (which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescence is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119:879-890) and the references cited therein. In one embodiment the method according to the present description is practiced using an electrochemiluminescent label.

Recently also Iridium-based ECL-labels have been described (WO2012107419).

### (c) Radioactive Labels Make Use of Radioisotopes (Radionuclides), Such as 3H, 11C, 14C, 18F, 32P, 35S, 64Cu, 68Gn, 86Y, 89Zr, 99TC, 111 In, 1231, 1241, 1251, 1311, 133Xe, 177Lu, 211At, or 131Bi.

(d) Metal-chelate complexes suitable as labels for imaging and therapeutic purposes are well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990). Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, ct al, J. Nucl. Mcd. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

### EXAMPLES

The invention will be merely illustrated by the following Examples. The said Examples shall, whatsoever, not be construed in a manner limiting the scope of the invention.

### Example 1: Diagnostic Performance of Biomarker FGFBP1 in Women with PCOS (Phenotype A) and Controls Determined by the Proximity Extension Assay (PEA) Technology Developed at Olink

As part of the measurements, 85 serum samples from human females were analyzed. The case group comprised 48 samples from patients diagnosed with PCOS (Phenotype A) according to the Rotterdam criteria. The control group included 37 samples from healthy women without PCOS. The concentration of the analytes was determined using the Proximity Extension Assay (PEA) technology developed at Olink. Briefly, a matched pair of antibodies, coupled to unique, partially complementary oligonucleotides, addresses each biomarker. Quantification is then performed by quantitative real-time PCR.

After sample dilution according to the manufacturer's protocol for each of the selected panels, the Olink protocol consists of three core steps: 1. Incubation, 2. Extension and amplification. 3. Detection. One μl of each sample was mixed with 3 μl of incubation mix in a 96 well plate. The incubation mix, additionally to the 92 antibody pairs, labeled with DNA oligonucleotides, contains also internal controls designed to monitor the three main steps of the Olink protocol (2 incubation controls, 1 extension control and 1 detection control). As external controls, 3 positive (inter-plate control) and 3 negative controls were included in the plate as well as 2 sample controls (pooled plasma samples). Samples were incubated overnight at +4° C. During this step the antibody pairs bind to the respective protein in the samples. When the incubation was complete, 96 μl of Extension mix were added to the samples. The plate was placed in a thermal cycler for hybridization and extension was performed by a DNA polymerase (50° C. 20 min, 95° C. 5 min (95° C. 30s, 54° C. 1 min, 60° C. 1 min) ×17, 10° C. hold). The DNA barcode was amplified by PCR. Finally, the amount of each DNA barcode was quantified by microfluidic qPCR. A 96.96 Dynamic Array™ Integrated Fluidic Circuit (IFC) was used according to the manufacturer's instructions. Seven point two μl of Detection mix were added to 2.8 μl of each sample, of these, 5 μl were transferred into the primed 96.96 Dynamic Array IFC left inlets. Five μl of primer solution were transferred into the primed 96.96 Dynamic Array IFC right inlets. Chip was loaded in the Fluidigm IFC Controller HX according to the manufacturer's instructions. The Olink Protein Expression 96×96 Program was ran in the Fluidigm Biomark™ Reader according to the manufacturer's instructions (50° C. 120 s, 70° C. 1800 s, 25° C. 600 s, 95° C. 300 s (95° C. 15 s, 60° C. 60 s)×35; with the following settings: application-Gene Expression; passive Reference-ROX; assay-Single probe; probes-FAM-MGB). The Ct values obtained from the qPCR were transformed into arbitrary unit called Normalized Protein expression (NPX, a relative quantification unit on log 2 scale) using the following equations:

### Extension Control:

$$Correction factor - {ddCt}_{Analyte} = {NPX}_{Analyte}$$

### Inter-plate Control:

$${dCt}_{Analyte} - {dCt}_{Inter - plate Control} = {ddCt}_{Analyte}$$

Adjustment against a correction factor:

$${Ct}_{Analyte} - {Ct}_{Extension Control} = {dCt}_{Analyte}$$

Quality control and normalization was achieved using the Olink NPX Manager software.

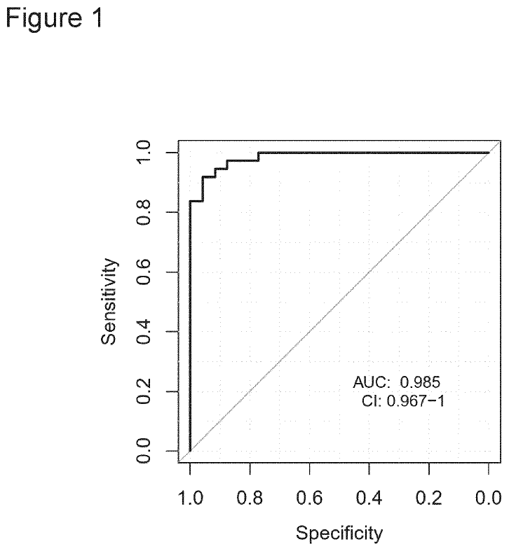

Receiver Operating Characteristic (ROC) curves were generated (FIG. 1). The model performance is determined by looking at the area under the curve (AUC). The best possible AUC is 1 while the lowest possible is 0.5. The ROC curve analysis illustrated an AUC of 0.985 (95% CI 0.967-1.0, FIG. 1), showing that FGFBP1 has high diagnostic accuracy for PCOS.

The diagnostic performance of FGFBP1 to distinguish between women with PCOS with the full blown phenotype A (cases) and healthy control subjects using ROC analysis is shown in Table 1 describing the AUC of the ROC curve analysis and the associated 95% confidence interval. The results were obtained using Olink proximity extension technology.

TABLE 1

| | AUC (area | 95% CI | N | |
|---|---|---|---|---|
| | under the | (confidence | (sample | Sample |
| Marker | curve) | interval) | size) | type |
| FGFBP1 | 0.985 | 0.967-1 | 85 | serum |

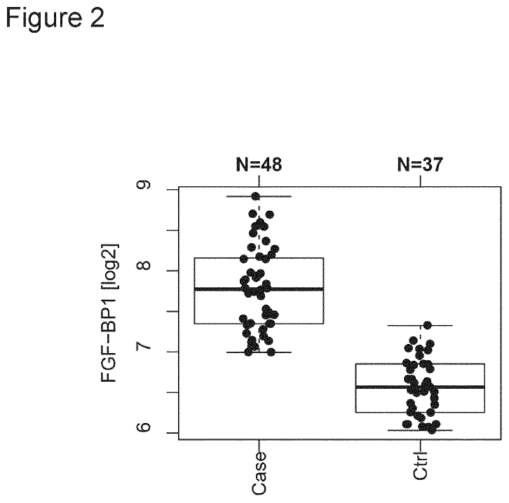

The data obtained by the Olink PEA technology were used to generate box and whisker plots for healthy controls and PCOS cases. The boxes include the median (middle quartile), the interquartile range (which represents the middle 50% of scores for the group), the upper quartile (75% of scores fall below the upper quartile) and the lower quartile (25% of scores fall below the lower quartile). The whiskers represent values that are 1.5 times the interquartile ranges. Serum FGFBP1 concentrations are increased in women with PCOS when compared to healthy controls (FIG. 2).

### Example 2: Diagnostic Performance of Biomarker FGFBP1 in Women with PCOS (Phenotypes a, B, C and D) and Controls Determined by ELISA Technology

Performance validation has been performed in a sample collective of 90 cases (serum samples from women with PCOS) and 44 controls (serum samples from healthy women).

The concentration of the analyte was determined by ELISA (enzyme-linked immunosorbent assay). The case group is composed of patients diagnosed with PCOS (30 phenotype A, 20 phenotype B, 20 phenotype C and 20 phenotype D) according to the Rotterdam criteria. The control group includes healthy women without PCOS.

The concentration of FGFBP1 in human serum was determined using the Human FGFBP1 ELISA kit from Sigma-Aldrich (catalog number: RAB1460). The kit is a solid-phase sandwich Enzyme-Linked Immunosorbent Assay (ELISA) designed to detect and quantify the level of human FGFBP1 in cell culture supernatants, plasma, and serum.

Human FGFBP1 antibody pre-coated plates are provided with the kit. Samples were measured in 75-fold dilution. After bringing all reagents to room temperature 100 μL of each sample and standard were added to the plate. Samples and standards were measured in duplicates. During 2.5 hours incubation at room temperature with gentle shaking, any FGFBP1 present was bound to the immobilized capture antibody on the microtiter plate. During the washing step (4×300 μL), unbound substances were removed from the plate before 100 μL of the diluted Detection Antibody was added to the wells. Following 1 h incubation with gentle shaking and another washing step (4×300 μL) to remove any unbound detection antibody, 100 μL of the prepared Streptavidin-HRP solution was added to the plate. Followed a 45 minutes incubation at room temperature with gentle shaking and a washing step (4×300 μL). After the last wash, 100 μL of TMB One-Step Substrate Reagent was added to the plate. The plate was incubated for 30 minutes at room temperature in the dark with gentle shaking. During the incubation the substrate turned blue. The color developed in proportion to the amount of FGFBP1 bound in the initial step. Color development was stopped by addition of 50 μL stop solution, the solution in the well changed from blue to yellow and color intensity was measured with a plate reader at 450 nm for detection and 570 nm for background subtraction. A seven point standard curve was obtained using 2.5-fold serial dilutions of the recombinant FGFBP1 in the Assay Diluent. The calibration curves were fitted using a 4-parameter nonlinear regression (Newton/Raphson) with no weighting.

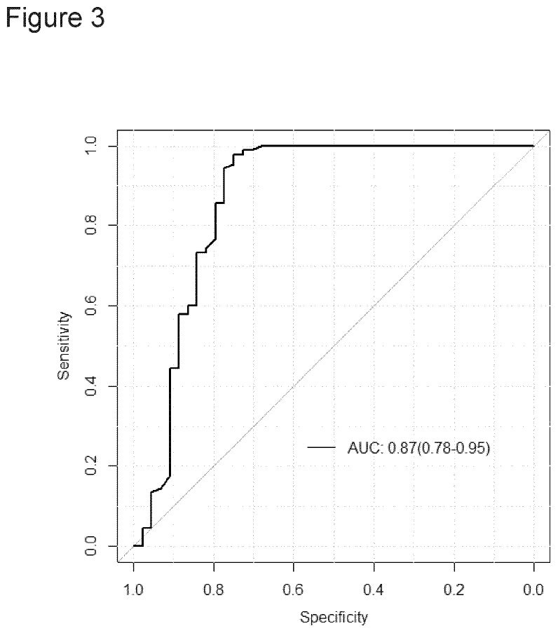

Receiver Operating Characteristic (ROC) curves were generated (FIG. 3). The model performance is determined by looking at the area under the curve (AUC). The best possible AUC is 1 while the lowest possible is 0.5. The ROC curve analysis for FGFBP1 for PCOS cases when all phenotypes were combined (phenotypes A-D) illustrated an AUC of 0.87 (95% CI 0.78-0.95), confirming the high diagnostic accuracy of FGFBP1 for PCOS (FIG. 3).

The diagnostic performance of FGFBP1 to distinguish between women with PCOS (cases, PCOS phenotypes A-D) and healthy control subjects using ROC analysis is shown in Table 2 describing the AUC of the ROC curve analysis and the associated 95% confidence interval. The results were obtained using ELISA immunoassay.

TABLE 2

| | AUC (area | 95% CI | N | |
|---|---|---|---|---|
| | under the | (confidence | (sample | Sample |
| Marker | curve) | interval) | size) | type |
| FGFBP1 | 0.87 | (0.78-0.95) | 134 | scrum |

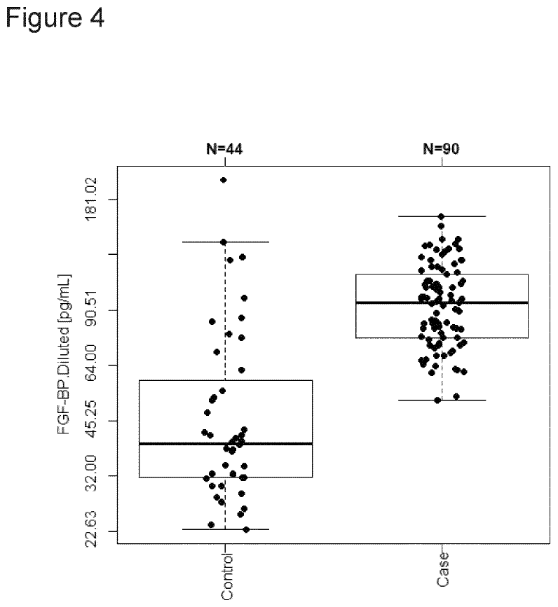

The data obtained by ELISA immunoassay were used to generate box and whisker plots for control and PCOS cases when all phenotypes were combined (phenotypes A-D). Serum FGFBP1 concentrations (pg/mL) are increased in women with PCOS when compared to healthy controls (FIG. 4).

Table 3 shows the diagnostic performance of FGFBP1 to distinguish between women with PCOS when separated by the different phenotypes A, B, C and D versus healthy control subjects. The results were obtained using ELISA immunoassay. AUC for each phenotype is reported in the table.

TABLE 3

| | Pheno- | Pheno- | Pheno- | Pheno- | N (sample | Sample |
|---|---|---|---|---|---|---|
| Marker | type A | type B | type C | type D | size) | type |
| FGFBP1 | 0.88 | 0.88 | 0.88 | 0.84 | 134 | serum |

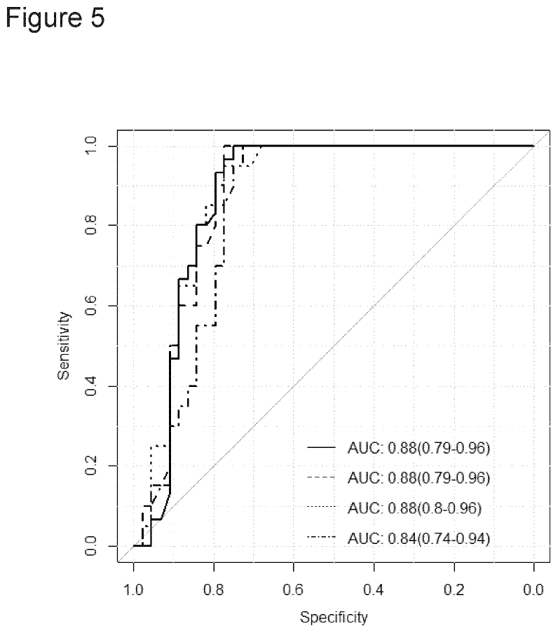

ROC curve analysis for FGFBP1 for PCOS cases separated by the different phenotypes versus healthy controls showed an AUC of 0.88 (95% CI 0.79-0.96), 0.88 (95% CI 0.79-0.96), 0.88 (95% CI 0.80-0.96) and 0.84 (95% CI 0.74-0.95), respectively for the phenotypes from A to D (FIG. 5). The results confirm the high diagnostic accuracy of FGFBP1 for PCOS.

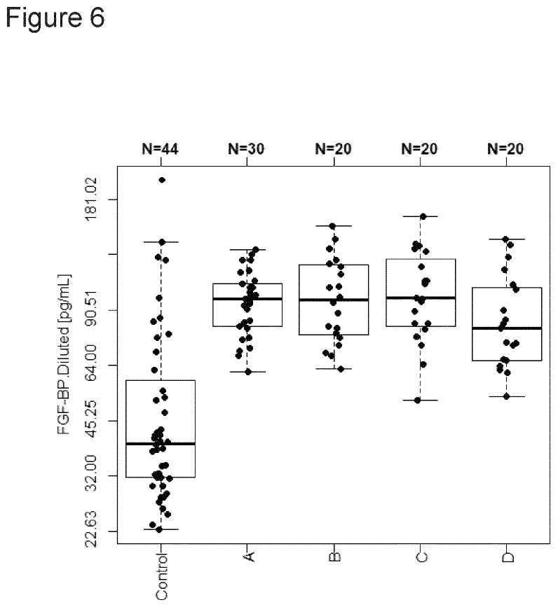

Serum FGFBP1 concentrations (pg/mL) in all the different PCOS phenotypes (Phenotype A-D) showed increased levels compared to healthy controls (FIG. 6, results obtained using ELISA immunoassay).

Table 4 shows the diagnostic performance of FGFBP1 in young women (age≤25), to distinguish young women with PCOS from young healthy control subjects when all phenotypes were combined (phenotypes A-D). The results were obtained using ELISA assays.

TABLE 4

| | AUC (area | 95% CI | N | |
|---|---|---|---|---|
| | under the | (confidence | (sample | Sample |
| Marker | curve) | interval) | size) | type |
| FGFBP1 | 0.84 | (0.61-1) | 32 | serum |

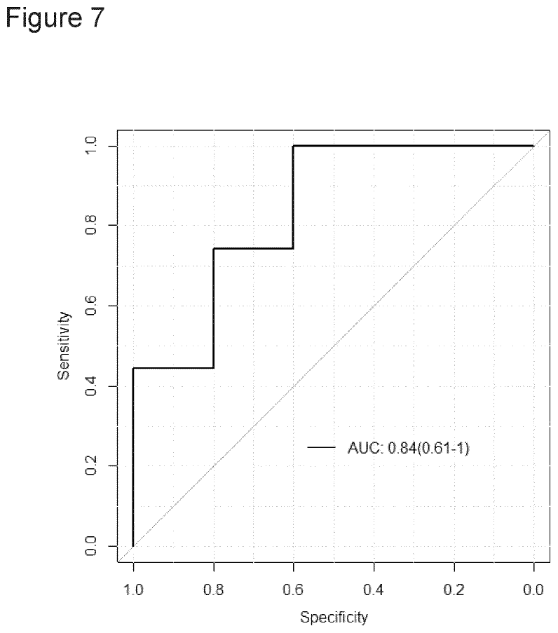

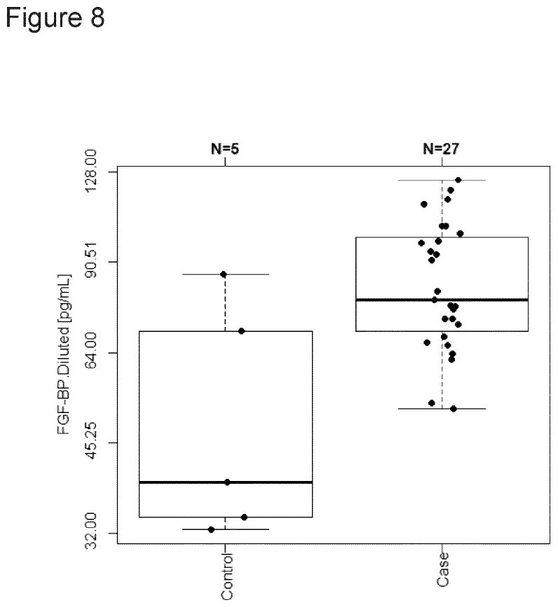

The ROC curve analysis for FGFBP1 for young PCOS cases (age≤25) when all phenotypes were combined (phenotypes A-D) illustrated an AUC of 0.84, confirming high diagnostic accuracy, for women that are 25 or younger, in discriminating PCOS cases from controls (95% CI 0.61-1, FIG. 7). When only young women were included in the analysis (age≤25) PCOS cases (all phenotypes A-D combined) showed increased serum FGFBP1 concentrations (pg/mL) compared to young controls (age≤25, FIG. 8).

The diagnostic performance of FGFBP1 to distinguish between young women with PCOS (age≤25) when separated by the different phenotypes A, B, C and D versus young healthy control subjects (age≤25) has been evaluated and results are reported in table 5 (AUC for PCOS phenotypes versus controls). The results were obtained using ELISA immunoassay.

TABLE 5

| | Pheno- | Pheno- | Pheno- | Pheno- | N (sample | Sample |
|---|---|---|---|---|---|---|
| Marker | type A | type B | type C | type D | size) | type |
| FGFBP1 | 0.93 | 0.85 | 0.84 | 0.63 | 32 | serum |

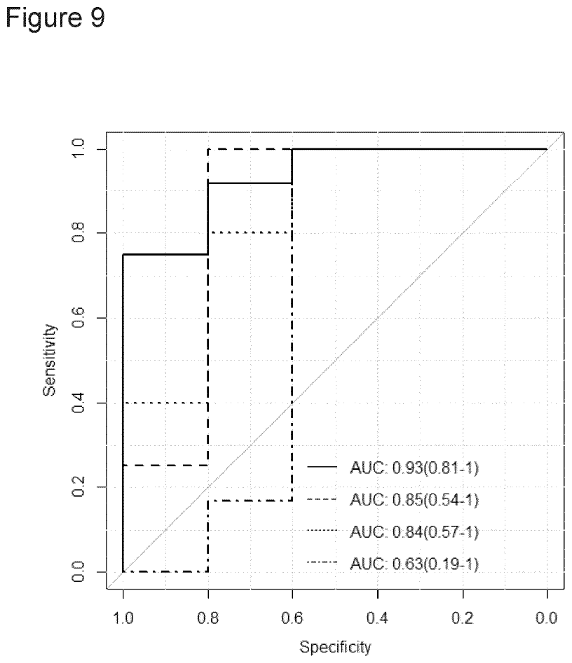

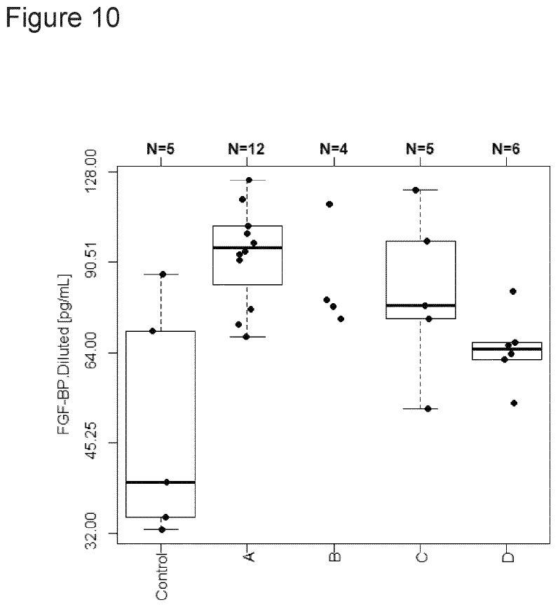

The ROC curve analysis for FGFBP1 for young PCOS cases (age≤25, phenotypes A-D) illustrated an AUC of 0.93 (95% CI 0.81-1), 0.85 (95% CI 0.54-1), 0.84 (95% CI 0.57-1), 0.63 (95% CI 0.19-1) for each phenotype respectively, confirming high diagnostic accuracy of FGFBP1 for PCOS, in the subgroup of women of age 25 or younger (FIG. 9). FGFBP1 concentrations were increased in all the different PCOS phenotypes (Phenotype A-D, age≤25) when compared to FGFBP1 concentrations in young healthy controls (age≤25, FIG. 10).

### Example 3: Diagnostic Performance of Biomarker FGFBP1 in Women with PCOS (Phenotypes A, B, C and D) and Controls Determined by ELISA Technology, in Different Age Groups

Performance validation has been performed in an additional sample collective of 240 cases (serum samples from women with PCOS) and 48 controls (serum samples from healthy women).

The concentration of the analyte was determined by ELISA (enzyme-linked immunosorbent assay). The case group is composed of patients diagnosed with PCOS (155 phenotype A, 5 phenotype B, 8 phenotype C and 72 phenotype D) according to the Rotterdam criteria, belonging to three different age groups: 15-<20 (n=70), 20-<25 (n=99), 25-<40 (n=71). The control group includes healthy women without PCOS.

The concentration of FGFBP1 in human serum was determined as described in example 2 using the Human FGFBP1 ELISA kit from Sigma-Aldrich (catalog number: RAB1460).

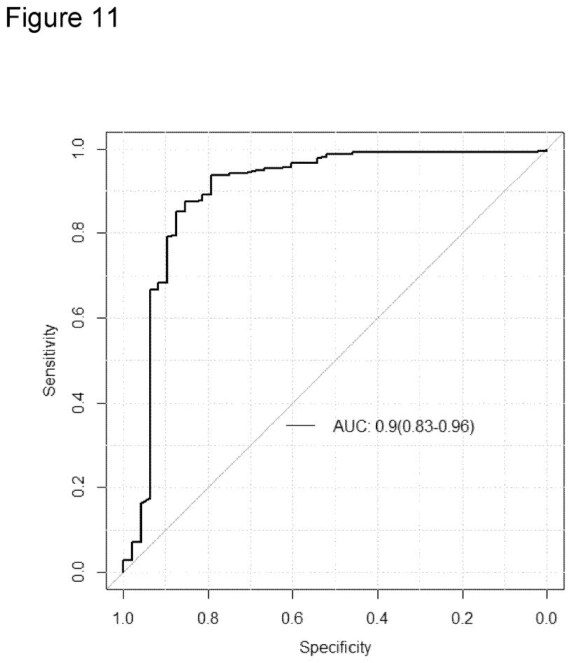

Receiver Operating Characteristic (ROC) curves were generated (FIG. 11). The model performance is determined by looking at the area under the curve (AUC). The ROC curve analysis for FGFBP1 for PCOS cases when all phenotypes were combined (phenotypes A-D) illustrated an AUC of 0.9 (95% CI 0.83-0.96), confirming the high diagnostic accuracy of FGFBP1 for PCOS (FIG. 11).

The diagnostic performance of FGFBP1 to distinguish between women with PCOS (cases, PCOS phenotypes A-D) and healthy control subjects using ROC analysis is shown in Table 6 describing the AUC of the ROC curve analysis and the associated 95% confidence interval. The results were obtained using ELISA immunoassay.

TABLE 6

| | AUC (area | 95% CI | N | |
|---|---|---|---|---|
| | under the | (confidence | (sample | Sample |
| Marker | curve) | interval) | size) | type |
| FGFBP1 | 0.90 | (0.83-0.96) | 288 | serum |

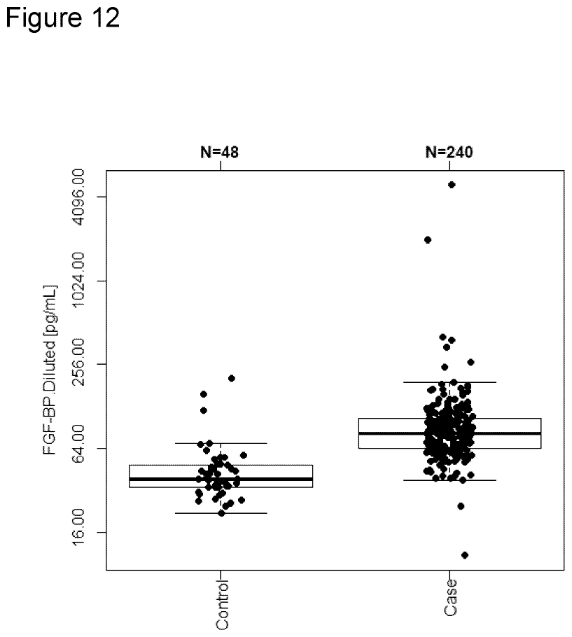

The data obtained by ELISA immunoassay were used to generate box and whisker plots for control and PCOS cases when all phenotypes were combined (phenotypes A-D). Serum FGFBP1 concentrations (pg/mL) are increased in women with PCOS when compared to healthy controls (FIG. 12).

Table 7 shows the diagnostic performance of FGFBP1 to distinguish between women with PCOS when separated by the different phenotypes A, B, C and D versus healthy control subjects. The results were obtained using ELISA immunoassay. AUC for each phenotype is reported in the table.

TABLE 7

| | Pheno- | Pheno- | Pheno- | Pheno- | N (sample | Sample |
|---|---|---|---|---|---|---|
| Marker | type A | type B | type C | type D | size) | type |
| FGFBP1 | 0.90 | 0.90 | 0.82 | 0.90 | 288 | serum |

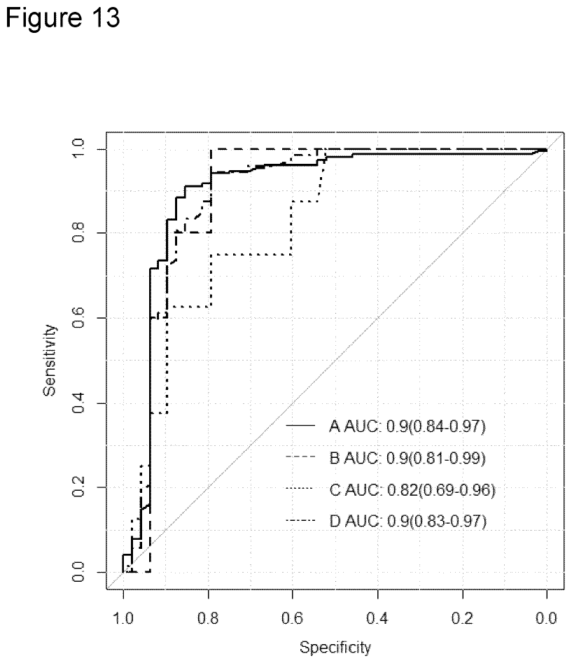

ROC curve analysis for FGFBP1 for PCOS cases separated by the different phenotypes versus healthy controls showed an AUC of 0.90 (95% CI 0.84-0.97), 0.90 (95% CI 0.81-0.99), 0.82 (95% CI 0.69-0.96) and 0.90 (95% CI 0.83-0.97), respectively for the phenotypes from A to D (FIG. 13). The results confirm the high diagnostic accuracy of FGFBP1 for PCOS.

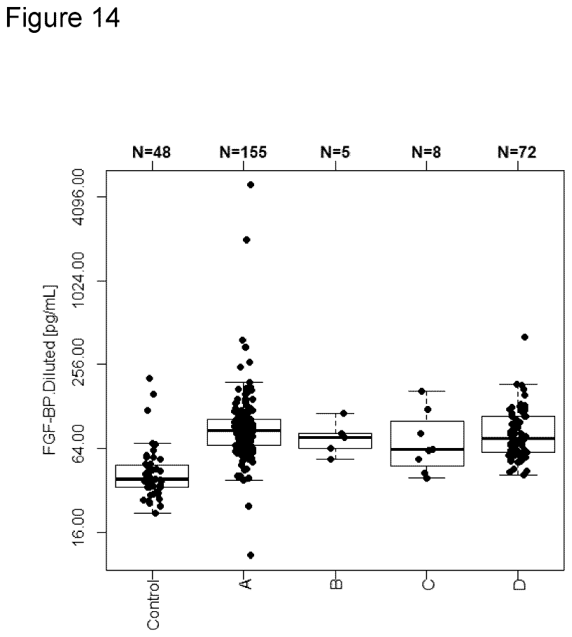

Serum FGFBP1 concentrations (pg/mL) in all the different PCOS phenotypes (Phenotype A-D) showed increased levels compared to healthy controls (FIG. 14, results obtained using ELISA immunoassay).

Table 8 shows the diagnostic performance of FGFBP1 in different age groups (15≤age<20, 20≤age<25, 25≤age<40), to distinguish women with PCOS from healthy control subjects when all phenotypes were combined (phenotypes A-D). The results were obtained using ELISA assays.

TABLE 8

| | 15 ≤ | 20 ≤ | 25 ≤ | N | |
|---|---|---|---|---|---|
| | age < | age < | age < | (sample | Sample |
| Marker | 20 | 25 | 40 | size) | type |
| FGFBP1 | 0.90 | 0.88 | 0.91 | 288 | serum |

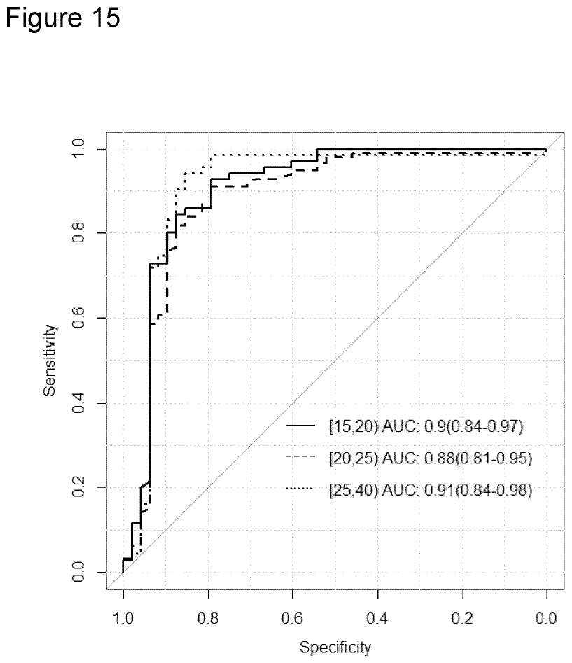

ROC curve analysis for FGFBP1 for PCOS cases separated by the different age groups versus healthy controls showed an AUC of 0.90 (95% CI 0.84-0.97), 0.88 (95% CI0.81-0.95) and 0.91 (95% CI 0.84-0.98), respectively for the age groups 15-5<20, 20-<25, 25-<40 (FIG. 15). The results confirm the high diagnostic accuracy of FGFBP1 for PCOS in all the different age groups.

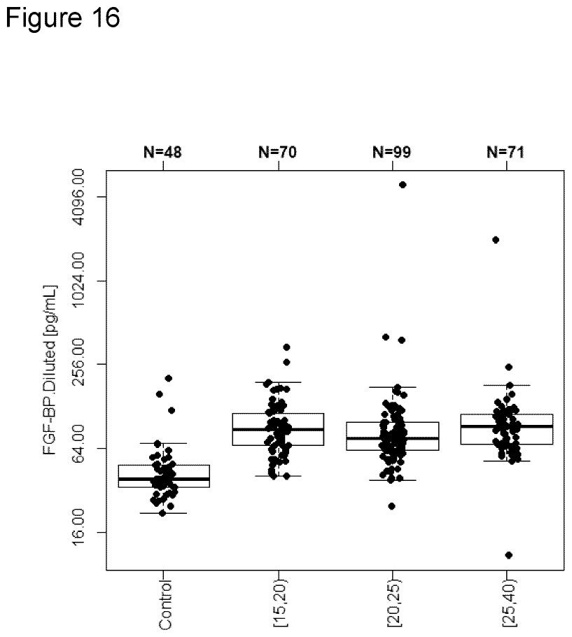

Increased serum FGFBP1 concentrations (pg/mL) in women with PCOS compared to controls were confirmed in all the different age groups (FIG. 16).

Considering the lack of reliable biomarkers for diagnosing PCOS especially in young women (age<25), a separate analysis was performed for the age group≥15 and <25.

Table 9 shows the diagnostic performance of FGFBP1 in young women (15≤age<25), to distinguish young women with PCOS from young healthy control subjects when all phenotypes were combined (phenotypes A-D). The results were obtained using ELISA assays.

TABLE 9

| | AUC (area | 95% CI | N | |
|---|---|---|---|---|
| | under the | (confidence | (sample | Sample |
| Marker | curve) | interval) | size) | type |
| FGFBP1 | 0.88 | (0.76-0.99) | 192 | serum |

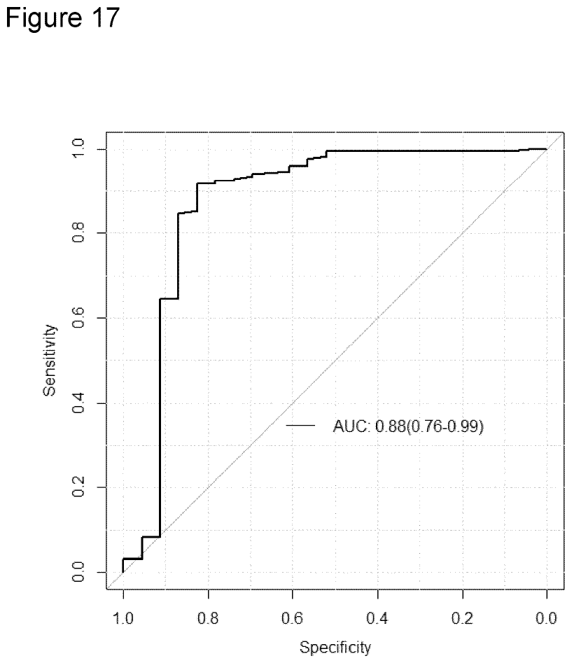

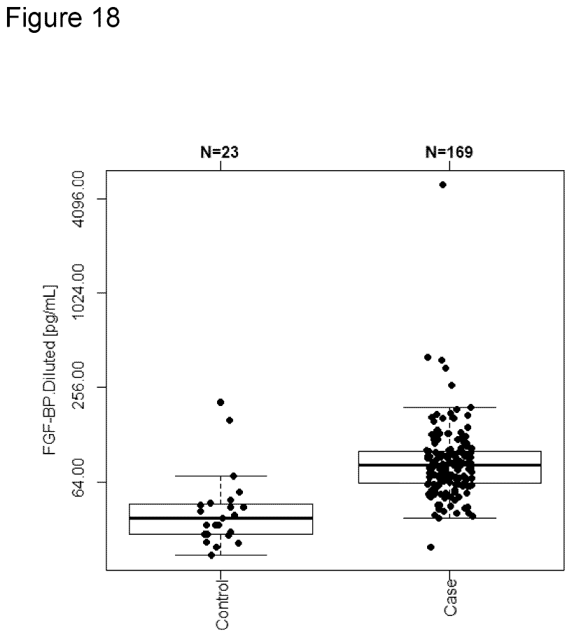

The ROC curve analysis for FGFBP1 for young PCOS cases (15≤age<25) when all phenotypes were combined (phenotypes A-D) illustrated an AUC of 0.88, confirming high diagnostic accuracy for 15-<25 year old women, in discriminating PCOS cases from controls (95% CI 0.76-0.99, FIG. 17). When only young women were included in the analysis (15≤age<25) PCOS cases (all phenotypes A-D combined) showed increased serum FGFBP1 concentrations (pg/mL) compared to young controls (15≤age<25, FIG. 18).

The diagnostic performance of FGFBP1 to distinguish between young women with PCOS (15≤age<25) when separated by the different phenotypes A, B, C and D versus young healthy control subjects (15≤age<25) has been evaluated and results are reported in table 10 (AUC for PCOS phenotypes versus controls). The results were obtained using ELISA immunoassay.

TABLE 10

| | Pheno- | Pheno- | Pheno- | Pheno- | N (sample | Sample |
|---|---|---|---|---|---|---|
| Marker | type A | type B | type C | type D | size) | type |
| FGFBP1 | 0.88 | 0.87 | 0.80 | 0.87 | 192 | serum |

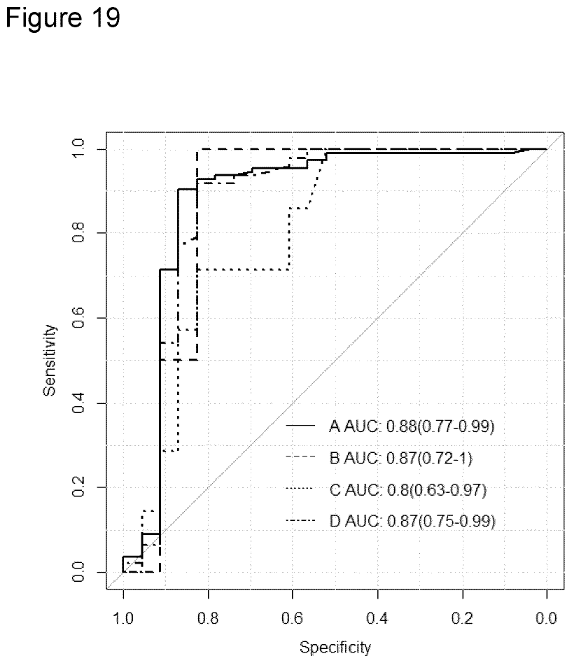

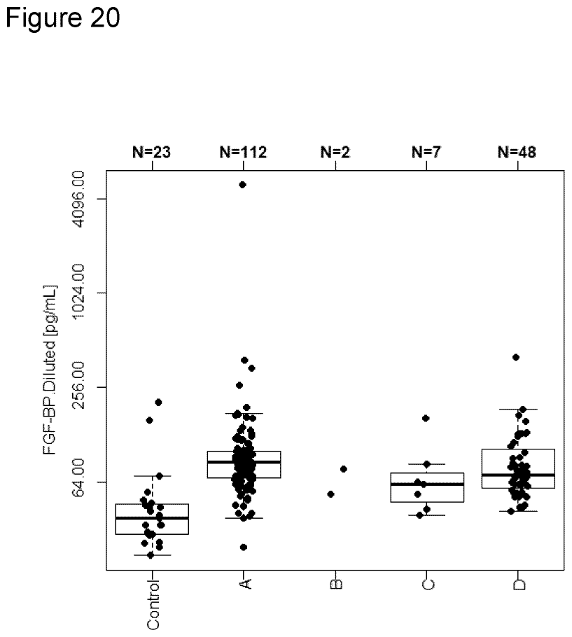

The ROC curve analysis for FGFBP1 for young PCOS cases (15≤age<25, phenotypes A-D) illustrated an AUC of 0.88 (95% CI 0.77-0.99), 0.87 (95% CI0.72-1), 0.80 (95% CI 0.63-0.97), 0.87 (95% CI 0.75-0.99) for each phenotype respectively, confirming high diagnostic accuracy of FGFBP1 for PCOS, in the subgroup of women of age≥15 and <25 (FIG. 19). FGFBP1 concentrations were increased in all the different PCOS phenotypes (Phenotype A-D, 15≤age<25), when compared to FGFBP1 concentrations in young healthy controls (15≤age<25, FIG. 20).

1. A method of assessing whether a subject has Polycystic Ovarian Syndrome (PCOS) or is at risk of developing PCOS, said method comprising:
(a) determining the amount or concentration of FGFBP1 in a sample of the subject; and
(b) comparing the determined amount or concentration to a reference.

2. A method of selecting a patient for therapy of PCOS, said method comprising:
(a) determining the amount or concentration of FGFBP1 in a sample of the subject; and
(b) comparing the determined amount or concentration to a reference.

3. A method for monitoring PCOS progression in a subject having PCOS or for monitoring response to treatment in a subject having PCOS, said method comprising:
(a) determining the level of FGFBP1 in a first sample of the subject;
(b) determining the level of FGFBP1 in a second sample of the subject which has been obtained after the first sample, and;
(c) comparing the level of FGFBP1 in the first sample to the level of FGFBP1 in the second sample; and
(d) monitoring progression in the subject suffering from PCOS or being treated for PCOS, based on the results of step c).

4. The method of claim 1, wherein an increased amount or concentration of FGFBP1 in the sample of the subject is indicative of the presence of PCOS in the subject.

5. The method of claim 1, wherein the sample is a blood, a serum or a plasma sample.

6. The method of claim 1, wherein PCOS is selected from the group consisting of PCOS of phenotype A, PCOS of phenotype B, PCOS of phenotype C and PCOS of phenotype D according to the Rotterdam scale.

7. The method of claim 1, wherein PCOS of phenotype A is detected.

8. The method of claim 1, wherein PCOS of phenotype B is detected.

9. The method of claim 1, wherein PCOS of phenotype C is detected.

10. The method of claim 1, wherein PCOS of phenotype D is detected.

11. The method of claim 1, wherein PCOS is detected in adolescents or young women.

12. The method of claim 1, wherein the subject suffers from oligo-anovulation, irregular cycles, hyperandrogenism, polycystic ovarian morphology, infertility, diabetes type 2, obesity, and/or psychological distress.

13. (canceled)

14. (canceled)

15. The method of claim 1, wherein the subject is a human.

16. The method of claim 2, wherein the therapy is a drug-based therapy of PCOS or lifestyle changes to control metabolic symptoms.

17. The method of claim 16, wherein the drug-based therapy of PCOS is selected from the group consisting of drugs for regulating periods, drugs for preventing or controlling diabetes, drugs for preventing or controlling high cholesterol, hormones or drugs to increase fertility, hormones to remove excess hair, and drugs to control acne.

18. The method of claim 1, wherein the determination of the amount or concentration of FGFBP1 comprises contacting the sample with an antibody, or antigen-binding fragment thereof, which specifically detects FGFBP1.

19. The method of claim 18, wherein the determination of the amount or concentration of FGFBP1 comprises performing an immunoassay, and wherein the immunoassay is selected from the group consisting of an enzyme linked immunosorbent assay (ELISA), an enzyme immunoassay (EIA), a radioimmunoassay (RIA), and an immuno assay based on detection of luminescence, fluorescence, chemiluminescence or electrochemiluminescence.

20. The method of claim 1, wherein step a) comprises:
i) incubating the sample of the subject with one or more antibodies specifically binding to FGFBP1, thereby generating a complex between the antibody and FGFBP1, and
ii) quantifying the complex formed in step i), thereby quantifying the amount or concentration of FGFBP1 in the sample of the subject.

21. The method of claim 20, wherein in step i), the sample is incubated with two antibodies specifically binding to FGFBP1.

22. The method of claim 21, wherein a sandwich will be formed comprising a first antibody to FGFBP1, FGFBP1 (analyte) and the second antibody to FGFBP1, wherein the second antibody is detectably labeled.
